(19) United States
(12) Patent Application Publication
(10) Pub. No.: US 2010/0249738 A1
(43) Pub. Date: Sep. 30, 2010

(54) ABSORBENT ARTICLE COMPRISING AT LEAST ONE VENTILATION COMPONENT

(75) Inventors: Maryam Tondkar, Hisings Backa (SE); Pia Kalentun, Torslanda (SE); Per Bergström, Goteborg (SE); Eva Strömbom, Molndal (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/744,552

(22) PCT Filed: Dec. 21, 2007
(86) PCT No.: PCT/SE07/51081

§ 371 Date: May 25, 2010

(51) Int. Cl. A61F 13/15

(52) U.S. Cl. 604/367; 604/385.23

(57) ABSTRACT

An absorbent article comprises a liquid impermeable backsheet and an absorbent core. The backsheet is not overlaid by the core in at least one margin section. The article also comprises at least one ventilation component constituted by a ventilation material. Each ventilation component is positioned overlaying the backsheet and exclusively localized within one or more margin sections. The thickness of the ventilation material measured at 0.5 kPa is at least 1 mm. The porosity of the ventilation material is >80%. The ratio between the thickness of the ventilation material measured at 5 kPa and the thickness of the ventilation material measured at 0.5 kPa is >0.5. Each ventilation component is arranged such that an associated spacing is created between the wearer's skin and the backsheet when the absorbent article is worn.

### TECHNICAL FIELD

The present invention relates to an absorbent article, such as a diaper, sanitary napkin, incontinence guard or panty liner, comprising one or more ventilation components, each ventilation component being constituted by a ventilation material and being exclusively localized within one or more margin sections of the absorbent article.

### BACKGROUND OF THE INVENTION

There are many different types of absorbent articles, such as diapers, sanitary napkins, incontinence guards and panty liners, known on the market today. The most important function of such absorbent articles is to absorb and contain body exudates, like urine, faeces and menstrual liquid. However, the absorbent articles need also be comfortable to wear.

One factor influencing the comfort of an absorbent article is the climate (i.e. the temperature and absolute humidity) within the absorbent article when used. A climate involving warmth and a relatively high absolute humidity may be experienced as uncomfortable to the wearer. In addition, this climate may result in a relatively high humidity in the space between the article and the wearer's skin, whereby a relatively high skin hydration may be produced, which may give rise to skin irritations and rashes. The risk of development of skin irritations and rashes is usually particularly high in the area around the waist and in the areas at the legs (e.g. at leg openings). This is due to the fact that the article usually is in a particularly close contact with the skin in the area around the waist and at the legs, whereby ventilation may be inhibited in these areas.

In order to improve the comfort and reduce the risk of skin irritations and rashes, many attempts have been made to reduce the humidity and temperature within absorbent articles and/or between an absorbent article and the wearer's skin by promoting ventilation. Attempts have been made to promote ventilation within one or more components of an absorbent article and/or between an absorbent article and the wearer's skin.

One way of promoting ventilation of an absorbent article has been to replace the commonly utilized liquid- and vapor-impermeable backsheet, which normally is arranged on that side of the article which faces away from the wearer during use, with a liquid-impermeable backsheet displaying vapor-permeability (i.e. breathability). For example, microporous films have been utilized for the replacement. However, construction of an absorbent article with a backsheet which simultaneously has sufficient liquid-impermeability and sufficient breathability has been difficult. In most cases, the backsheet has had too poor breathability because the liquid-impermeability of the backsheet was given priority over the breathability. In those cases where the breathability was given priority, liquid penetration through the breathable backsheet has occurred.

Another way of promoting ventilation of an absorbent article is described in GB 2308814. That document describes an absorbent article comprising a humidity transfer area in at least one waistband section. A vapour permeable panel is formed in the backsheet in each humidity transfer area. In addition, the absorbent article comprises a humidity transfer material, which extends over the core and into at least one waistband section such that it overlies at least one humidity transfer area. Water vapour can move from the crotch section through the humidity transfer material to the humidity transfer area(s) in the waistband section(s). Once in a humidity transfer area, water vapour can be transferred out through the vapour permeable panel. However, since the humidity transfer material extends both over the core and the vapour permeable panel, there is a risk that liquid during the acquisition process, i.e. during transfer of liquid from the topsheet to the core, may fill up in the humidity transfer material and be transferred via the humidity transfer material to the vapour permeable panel, where it may leak out of the article. Thus, there is a leakage risk associated with the ventilation arrangement in GB 2308814. In addition, there is also a risk associated with the ventilation arrangement in GB 2308814 that the humidity transfer material may be at least temporarily occluded due to transfer of liquid from the core into the humidity transfer material. In case the humidity transfer material is occluded by liquid, the ventilation function thereof may be inhibited.

Furthermore, one way of promoting ventilation between an absorbent article and the wearer's skin is disclosed in WO 01/97739. This document discloses an absorbent article having air accumulators which extend along the leg openings and which are connected to bellows. The air accumulators are made of soft, resilient material and act to provide airflow towards the wearer's skin through a plurality of apertures. However, the arrangement with bellows and air accumulators is relatively expensive to produce and difficult to handle in a conventional machine for manufacturing absorbent articles.

Thus, there still exists a need for an improved way of promoting ventilation of an absorbent article in order to improve the comfort and reduce the risk of development of skin irritations and rashes.

### SUMMARY OF THE INVENTION

Accordingly, one object of the present invention is to provide an improved absorbent article, such as a diaper, sanitary napkin, incontinence guard or panty liner, said article having longitudinal (L) and transverse (T) directions and comprising:
a front end section, a rear end section and a crotch section interconnecting said end sections;
a front end edge, a rear end edge and two side edges;
a liquid impermeable backsheet;
an absorbent core positioned overlaying a portion of said backsheet;
at least one margin section, whereby said at least one margin section is located adjacent to at least one of said edges, whereby said backsheet is not overlaid by said absorbent core in said at least one margin section;
at least one ventilation component constituted by a ventilation material, said at least one ventilation component being positioned overlaying said backsheet and arranged such that it is exclusively localized within one or more margin sections.

This object has been achieved by the fact that:
the thickness of the ventilation material measured at 0.5 kPa is at least 1 mm, preferably at least 3 mm, more preferably at least 7 mm, most preferably 10 mm,
the porosity of the ventilation material is >80%, preferably >90%, most preferably >95%,
the ratio between the thickness of the ventilation material measured at 5 kPa and the thickness of the ventilation material measured at 0.5 kPa is >0.5, preferably >0.75, most preferably >0.95, and
said at least one ventilation component is arranged such that an associated spacing is created between the wearer's skin and the backsheet when the absorbent article is worn,
whereby passage of gas into, within and out of said at least one ventilation component is allowed, thus promoting ventilation of the absorbent article.

Preferred embodiments are listed in the dependent claims.

Still other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures described herein.

### DEFINITIONS

The term “absorbent article” refers to a product that is placed against the skin of the wearer to absorb and contain body exudates, like urine, faeces and menstrual liquid. The invention mainly refers to disposable absorbent articles, which means articles that are not intended to be laundered or otherwise restored or reused as an absorbent article after use. Examples of disposable articles include feminine hygiene products such as sanitary napkins, panty liners and sanitary panties; diapers and pant diapers for infants and incontinent adults; incontinence pads, and the like.

The term “ventilation” is used to describe movement of air (including warm air and moist air) or vapour into, within and/or out of an absorbent article. In the present context, the term “ventilation” is used to describe ventilation within an absorbent article, i.e. internally within one or more components of an article, ventilation between the article and the wearer's skin, and/or ventilation out of the article. Ventilation may be active (in that movement of air or vapour is promoted or restricted by bodily movements or arrangements such as valves or pumps) or passive (in that movement of air or vapour is not promoted or restricted, and is free to move in any direction of the article). Ventilation differs from simple breathability of an article, in that ventilation involves the movement of air or vapour in bulk. Convection is one form of ventilation. Natural convection (also called free convection) is spontaneous, whereby movement of air or vapour is driven by temperature differences between different regions (i.e. warm air or vapour rises upwards). Thus, air or vapour may be moved between different regions of an absorbent article (e.g. the inside and the outside) due to natural convection. Forced convection is not spontaneous. It may be achieved by means of e.g. a pumping effect due to arrangements such as pumps or fans. In addition, the pumping effect may be produced due to bodily movements.

In the following, the word “gas” is used to describe air, vapour and moist air. “Vapour” generally refers to water vapour.

### BRIEF DESCRIPTION OF THE DRAWINGS

In the drawings, wherein like reference characters denote similar elements throughout the several views:

FIG. 1a is a plan view of a first embodiment of an absorbent article according to the invention, seen from the wearer-facing side;

FIG. 1b is a side view of the first embodiment shown in FIG. 1a, seen from the front end edge;

FIG. 1c shows a cross-section of the first embodiment according to line I-I in FIG. 1a when the article is worn by a wearer;

FIG. 1d is a plan view of a variation of the first embodiment, seen from the garment-facing side;

FIG. 2 is a plan view of a second embodiment of an absorbent article according to the invention, seen from the wearer-facing side;

FIG. 3a is a plan view of a third embodiment of an absorbent article according to the invention, seen from the wearer-facing side;

FIG. 3b is a plan view of a variation of the third embodiment, seen from the garment-facing side;

FIG. 4 is a plan view of a fourth embodiment of an absorbent article according to the invention, seen from the wearer-facing side;

FIG. 5a is a plan view of a fifth embodiment of an absorbent article according to the invention, seen from the wearer-facing side;

FIG. 5b is a side view of the fifth embodiment shown in FIG. 5a, seen from the front end edge;

FIG. 5c shows a cross-section of the fifth embodiment according to line II-II in FIG. 5a when the article is worn by a wearer;

FIG. 6 is a side view of a sixth embodiment of an absorbent article according to the invention, seen from a side edge;

FIG. 7a is a plan view of a seventh embodiment of an absorbent article according to the invention, seen from the wearer-facing side;

FIG. 7b shows a cross-section of the seventh embodiment according to line in FIG. 7a when the article is worn by a wearer;

FIG. 8 is a plan view of an eighth embodiment of an absorbent article according to the invention, seen from the wearer-facing side; and

FIG. 9 shows a cross-section of a ninth embodiment when the article is worn by a wearer.

### DETAILED DESCRIPTION OF PREFERRED EMBODIMENTS

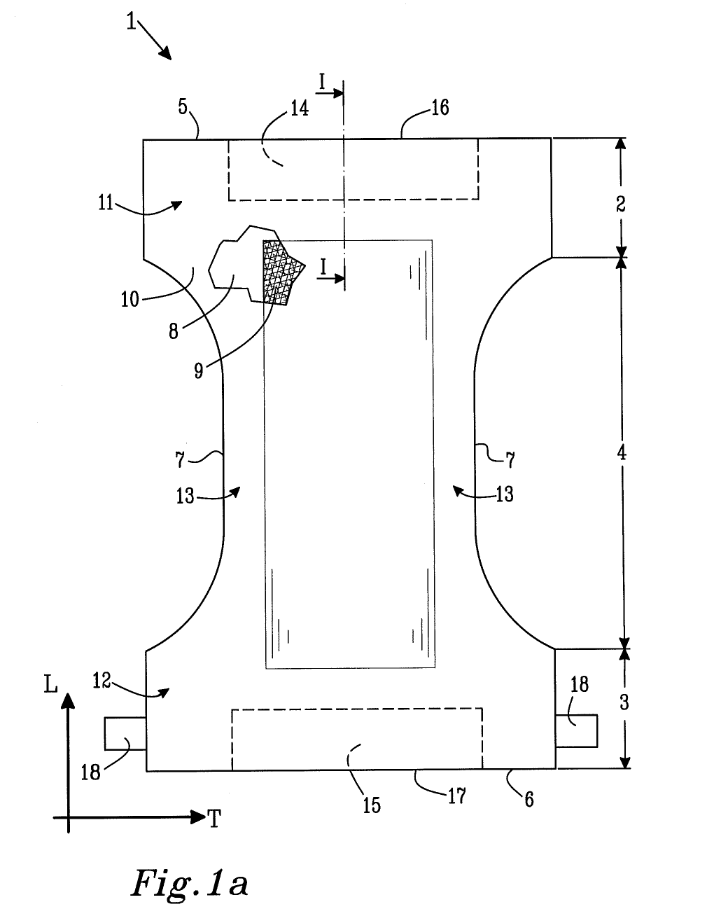

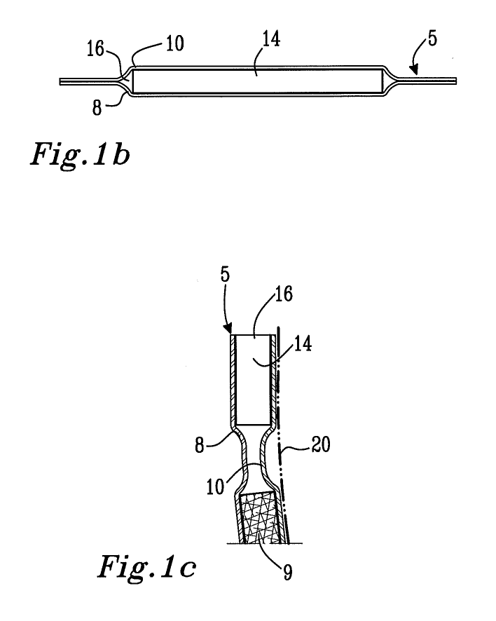

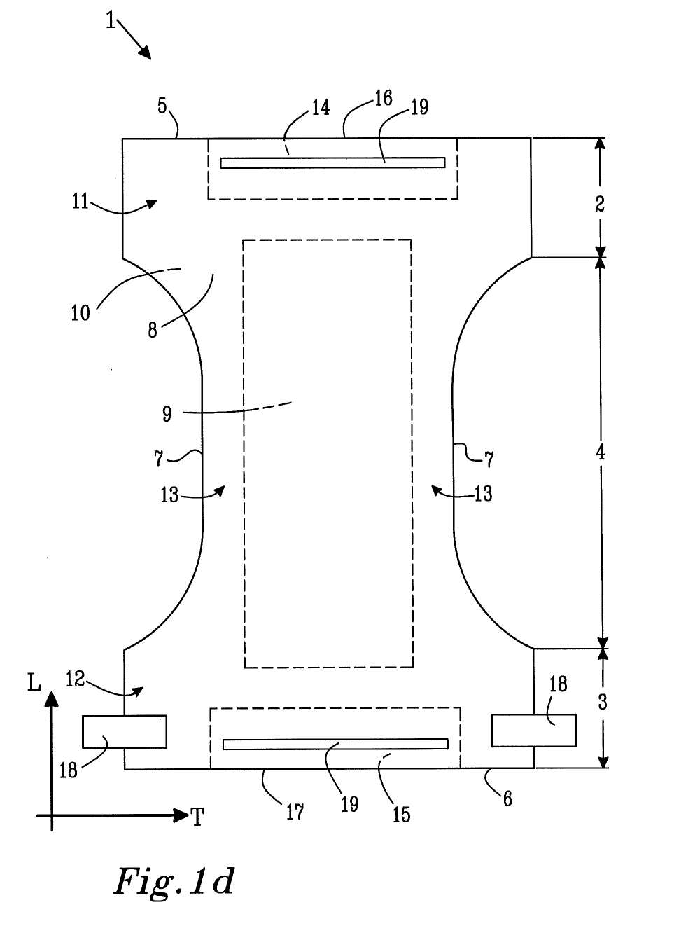

The invention will be described in further detail in the following, with reference to the accompanying figures. As mentioned previously, the invention concerns an absorbent article, such as a diaper, sanitary napkin, incontinence guard, panty liner or the like. FIG. 1a is a plan view of a first embodiment of an absorbent article 1 according to the invention, in this case a diaper, seen from the wearer-facing side. The absorbent article 1 has longitudinal (L) and transverse (T) directions as illustrated—the longitudinal (L) direction being along the longitudinal (longest) axis of symmetry, and the transverse direction being perpendicular to this. Furthermore, the absorbent article 1 has a front end section 2, a rear end section 3 and a crotch section 4 interconnecting the end sections 2, 3. The front and rear end sections 2, 3 are the sections of the absorbent article 1 which are intended to substantially extend over the wearer's front and rear abdominal regions, respectively, i.e. the sections which are located closest to the waist of the wearer, during use. In the diaper 1 shown in FIG. 1a, the front and rear end sections 2, 3 may be denoted as front and rear waistband sections, respectively. The crotch section 4 is the section of the absorbent article 1 which is intended to be located between the wearer's legs, i.e. to extend over the wearer's crotch region, during use and is the section of the absorbent article 1 which during normal use is expected to be the first to be wetted by discharged body fluid.

The absorbent article 1 comprises also a front end edge 5, a rear end edge 6 and two side edges 7. In FIG. 1a, the end edges 5, 6 are shown as being essentially straight in the transverse (T) direction and are intended to form a waist opening during use. The two side edges 7 form the remainder of the edge parts of the article 1. Thus, each side edge 7 runs from the front end edge 5 to the rear end edge 6 on one respective side of the article 1, i.e. each side edge 7 is formed by all edge parts of the article 1 on one side of the article 1 between the front end edge 5 and the rear end edge 6. However, the front and rear end edges 5, 6 need not be straight, i.e. they may be at least partly curved. Thus, there may be a “smooth” transition from an end edge 5, 6 to a side edge 7 such that the article 1 has rounded corners. In this case, the transition between an end edge 5, 6 and a side edge 7 is defined as the point on the edge of the article at which the rate of curvature of the end edge 5, 6 with respect to the transverse (T) direction is greatest.

Furthermore, the absorbent article 1 comprises a liquid impermeable backsheet 8 and an absorbent core 9 positioned overlying a portion of the backsheet 8. In the present context, the terms “overlying” and “overlies” mean that the absorbent core 9 has such a position that it lies over the backsheet 8 when the absorbent article 1 has the orientation shown in FIG. 1a, i.e. when the article 1 is seen from the wearer-facing side. However, other components may be sandwiched between the backsheet 8 and the absorbent core 9. In addition, the first embodiment of the absorbent article 1 comprises a topsheet 10, which is liquid permeable in that both liquids and gases can pass through it. The topsheet 10 is generally coextensive with the backsheet 8 and overlies the absorbent core 9. Thus, the absorbent core 9 is positioned between the backsheet 8 and the topsheet 10 in the first embodiment.

The backsheet 8 and the topsheet 10 extend outside the edges of the absorbent core 9 and are connected (sealed) to each other within the projecting portions thereof, e.g. by gluing or welding or by heat or ultrasound. The backsheet 8 and/or the topsheet 10 may be further attached to the absorbent core 9 by any known method in the art, such as by means of adhesive, heat bonding, etc. The absorbent core 9 may also be attached to the backsheet 8 and/or topsheet 10 in certain regions only.

The backsheet 8 may consist of a thin plastic film, e.g. a polyethylene or polypropylene film, a nonwoven material coated with a liquid impervious material, a hydrophobic nonwoven material which resists liquid penetration, or a laminate of plastic films and nonwoven materials. The backsheet 8 may be breathable so as to allow vapour to escape from the absorbent core 9, while still preventing liquids from passing through the backsheet material. The backsheet 8 may be elastic. The material of the backsheet may be different in different regions of the backsheet.

The absorbent core 9 may be of any conventional kind. Examples of commonly occurring absorbent materials are cellulosic fluff pulp, tissue layers, airlaid cellulose material, highly absorbent polymers (so called superabsorbents), absorbent foam materials, absorbent nonwoven materials or the like. It is common to combine cellulosic fibers with superabsorbents in an absorbent core. It is also common to have absorbent cores comprising layers of different material with different properties with respect to liquid acquisition capacity, liquid distribution capacity and storage capacity. This is well-known to the person skilled in the art and does therefore not have to be described in detail. The thin absorbent cores, which are common in today's absorbent articles, often comprise a compressed mixed or layered structure of cellulosic fibres and superabsorbent material. The size and absorbent capacity of the absorbent core may be varied to suit different uses such as sanitary napkins, pantiliners, adult incontinence pads and diapers, baby diapers, pant diapers, etc.

The liquid permeable topsheet 10 may comprise apertures through which liquid can permeate, or alternatively, liquid may permeate through the spaces between individual fibres. It may be any material used for this purpose, for example a nonwoven material, such as a spunbond material of continuous filaments, a meltblown material, a thermobonded fibrous web such as a carded fibrous web, a hydroentangled material, a wetlaid material, etc. The topsheet 10 may comprise many different types of fibres. For example, natural fibres such as wood pulp or cotton fibres, jute, wool and hair fibres may be used. Man-made fibres, such as e.g. polyester, viscose, nylon, polypropylene, and polyethylene may also be used, polypropylene and polyester being preferred. Mixtures of different fibres types may also be used, e.g. a 50/50 mix of polyester and viscose. Bicomponent fibres or binder fibres may also be used. The topsheet may also be a layer of so called tow fibres bonded in a bonding pattern, as e.g. disclosed in EP-A-1 035 818, or a perforated plastic film. The materials suited as top sheet materials should be soft and non-irritating to the skin and intended to be readily penetrated by body liquid, e.g. urine or menstrual liquid. The topsheet material may be different in different parts of the absorbent article.

The topsheet 10 may be treated with a chemical agent to improve one or more of its properties. For example, treatment of the topsheet with surfactants will make it more liquid-permeable. Treatment of the topsheet with a lotion, e.g. as described in EP1227776 provides a softer, more comfortable feel to the wearer, and improved skin properties.

Furthermore, the topsheet 10 may comprise at least two separate but interconnected layers. Each layer may comprise the same materials or may comprise different materials with different properties as regards e.g. strength, stiffness, liquid or gas permeability. Each layer may also be a laminate of two or more sub-layers.

Since the absorbent core 9 overlies a portion of the backsheet 8 only, there are sections of the absorbent article 1 in the L-T plane in which the backsheet 8 is not overlaid by the absorbent core 9, i.e. there are absorbent core free sections. In the present context, a section of the absorbent article 1 in the L-T plane in which the backsheet 8 is not overlaid by the absorbent core 9 is denoted as a “margin section”. Each margin section is located adjacent to at least one of the front end edge 5, the rear end edge 6 and the side edges 7. In the present application the term “first margin section” means a margin section located in the front end section 2. A first margin section may be located adjacent to the front end edge 5 only, or adjacent to the front end edge 5 as well as portions of the two side edges 7 located in the front end section 2. Alternatively, a first margin section may be located adjacent to the front end edge 5 as well as portions of one of the side edges 7 located in the front end section 2. However, a first margin section need not be located adjacent to the front end edge 5, but may solely be located adjacent to portions of one of the side edges 7 located in the front end section 2. The term “second margin section” means a margin section located in the rear end section 3. A second margin section may be located adjacent to the rear end edge 6 only, or adjacent to the rear end edge 6 as well as portions of the two side edges 7 located in the rear end section 3. Alternatively, a second margin section may be located adjacent to the rear end edge 6 as well as portions of one of the side edges 7 located in the rear end section 3. However, a second margin section need not be located adjacent to the rear end edge 6, but may solely be located adjacent to portions of one of the side edges 7 located in the rear end section 3. The term “third margin section” means a margin section located in the crotch section 4. A third margin section is located adjacent to portions of one of the side edges 7 located in the crotch section 4.

The first embodiment of the absorbent article 1 shown in FIG. 1a comprises one first margin section 11, one second margin section 12 and two third margin sections 13. More specifically, as is schematically illustrated in FIG. 1a, in the first embodiment the absorbent core 9 has such a size and shape in relation to the size and shape of the backsheet 8 and such a position on the backsheet 8 that one first margin section 11 is formed in the front end section 2, one second margin section 12 is formed in the rear end section 3 and one third margin section 13 is formed on each side of the absorbent core 9, i.e. one third margin section 13 is formed at each side edge 7, in the crotch section 4. Thus, in the first embodiment the first margin section 11 is constituted by the portion of the absorbent article 1 in the front end section 2 in which the backsheet 8 is not overlaid by the absorbent core 9. The first margin section 11 is positioned adjacent to the front end edge 5 as well as the portions of the side edges 7 located in the front end section 2. Likewise, the second margin section 12 is constituted by the portion of the absorbent article 1 in the rear end section 3 in which the backsheet 8 is not overlaid by the absorbent core 9. The second margin section 12 is positioned adjacent to the rear end edge 6 as well as the portions of the side edges 7 located in the rear end section 3. Each third margin section 13 is constituted by the portion of the absorbent article 1 at one side edge 7 in the crotch section 4 in which the backsheet 8 is not overlaid by the absorbent core 9. Each third margin section 13 is positioned adjacent to one of the side edges 7 in the crotch portion 4.

Furthermore, the first embodiment of the absorbent article 1 comprises two ventilation components; a first ventilation component 14 and a second ventilation component 15. Each ventilation component 14, 15 is constituted by (i.e. made of) a ventilation material. Thus, a ventilation material forms the ventilation components 14, 15. The first and second ventilation components 14, 15 are positioned overlaying the backsheet 8. In the present context the terms “overlying” and “overlies” mean that each ventilation component 14, 15 has such a position that it lies over the backsheet 8 when the article 1 has the orientation shown in FIG. 1a, i.e. when the article 1 is seen from the wearer-facing side. However, other components may be sandwiched between the backsheet 8 and the ventilation components 14, 15. More specifically, in the first embodiment the ventilation components 14, 15 are positioned between the backsheet 8 and the topsheet 10. Furthermore, the first ventilation component 14 is arranged such that it is exclusively localized within, i.e. comprised in, the first margin section 11 and the second ventilation component 15 is arranged such that it is exclusively localized within, i.e. comprised in, the second margin section 12. Thus, the first and second ventilation components 14, 15 do not overlap with, or extend over, the absorbent core 9, i.e. the ventilation material is only found in the ventilation components 14, 15 and does not extend over the absorbent core 9. The ventilation components 14, 15 may be attached to the backsheet 8 and/or topsheet 10 in one or more regions by any known method in the art, such as by means of adhesive, heat bonding, etc.

In the present application, the terms “first ventilation component” and “second ventilation component” mean a ventilation component exclusively localized within a first and a second margin section, respectively.

The ventilation material of the first and second ventilation components 14, 15 is a material that is particularly suitable for promoting ventilation of the absorbent article 1, i.e. the ventilation material has such characteristics that ventilation is promoted. Consequently, the ventilation material has such characteristics that gas flow into, within and out of the ventilation material, i.e. the ventilation components 14, 15, is allowed. More specifically, the thickness (caliper) of the ventilation material is at least 1 mm, preferably at least 3 mm, more preferably at least 7 mm, most preferably at least 10 mm as measured at 0.5 kPa in accordance with the thickness measurement method described below. The porosity of the ventilation material is >80%, preferably >90%, most preferably >95% as determined by the porosity determination method described below. Furthermore, the ratio between the thickness (caliper) of the ventilation material measured at 5 kPa and the thickness of the ventilation material measured at 0.5 kPa is >0.5, preferably >0.75, most preferably >0.95, whereby the thickness at 5 kPa and 0.5 kPa is measured by the thickness measurement method described below.

The ventilation material of the ventilation components 14, 15 is oriented such that the extension of the ventilation components 14, 15 in the Z-direction (i.e. the direction orthogonal to the longitudinal and transverse directions) of the article 1 corresponds to the thickness of the ventilation material. Thus, the thickness of the ventilation components 14, corresponds to the thickness of the ventilation material.

The fact that the thickness of the ventilation material is ≧1 mm, as mentioned above, implies that the thickness of the ventilation components 14, 15, i.e. their extension in the Z-direction, is ≧1 mm. The thickness of the ventilation components 14, 15 implies that each ventilation component 14, 15 create a spacing between the backsheet 8 and the wearer's skin when the article 1 is worn. In addition, the mentioned porosity of the ventilation material implies that the ventilation material has a relatively open structure with a relatively high degree of voids/pores, thus promoting gas flow into, within and out of the ventilation components 14, 15 (i.e. the created spacings). The gas flow may occur in all directions, i.e. in the transverse (T) direction, the longitudinal (L) direction or the Z-direction.

Furthermore, the fact that the ratio between the thickness of the ventilation material measured at 5 kPa and the thickness measured at 0.5 kPa is >0.5, as mentioned above, helps the ventilation components 14, 15 to maintain a relatively open structure when subjected to physical pressure, e.g. when a person wearing the article sits or lies down. This means that ventilation, i.e. gas flow into, within and out of the ventilation components 14, 15, is allowed both when the ventilation components 14, 15 are subjected to physical pressure according to the above and when not.

The extension of each of the first and second ventilation components 14, 15 in the longitudinal (L) and transverse (T) directions, respectively, of the absorbent article 1 depends on the size of the article 1, i.e. it depends on the type of the article 1. Thus, the extension of the ventilation components 14, 15 may differ between e.g. incontinence products for adults and diapers for premature babies. However, each of the first and second ventilation components 14, 15 may extend, for example, about 1-10 cm in the longitudinal (L) direction of the article 1, i.e. each may have a maximum extension in the longitudinal (L) direction of about 1-10 cm. The extension of the first and second ventilation components 14, 15 in the transverse (T) direction of the absorbent article 1 may be, for example, from about 1 cm and up to the width of the absorbent article 1 in the transverse (T) direction. However, concerning dimensions of the first and second ventilation components 14, 15, the thickness of the ventilation components 14, 15 is the most important characteristic in order to achieve the inventive concept.

The ventilation material may be, for example, a fibre- or yarn-based material, foam or corrugated structure. Examples of fibre-based materials are knitted or woven fabrics and nonwovens. Knitted (e.g. warp knitted, circular weft knitted or flat weft knitted) or woven spacer fabric materials are particularly useful according to the present invention, warp knitted spacer fabrics being the most common type. A “spacer fabric” is a 3D textile structure which comprises two fabric layers which are connected by cross threads, i.e. it comprises two distinctive layers of fabrics joined together by a connecting layer. The fibre composition of the two fabric layers may be the same or different. Likewise, the structure of the two fabric layers may be the same or different. Furthermore, nonwoven materials of 3D-type, such as stitch-bonded spacer fabrics or resilient loft materials can also be used as the ventilation material, as well as plain nonwoven materials. However, the latter ones normally need to be used in combination with a distance giving material having a certain thickness. In such constructions, the distance giving material is positioned between two layers of plain nonwoven webs and stabilized by any suitable means to keep the materials together and bring integrity to the formed laminate. The distance-giving material may for instance be a net, a fibrous material, a folded sheet/web material or foam material as long as the distance-giving material together with the two outer nonwoven layers present appropriate porosity, thickness and compression resistance allowing sufficient gas flow through the ventilation component. Suitable nonwoven materials are spunlaid, meltblown, carded, air-laid, wet-laid or layered combinations thereof such as SMS (spunlaid-meltblown-spunlaid). The nonwoven materials may be bonded using any conventional techniques well-known to those skilled in the art. The fibres or yarns forming the fibre-based materials may be of synthetic type such as polypropylene, polyethylene, polyesters, polyurethanes and polyamides. The fibre- or yarn-based materials can also be made of, or partly contain, cellulose-based fibres such as viscose, rayon and lyocell or natural fibres such as cotton and flax, or fibres from animal sources such as wool and silk.

A textile is a flexible material comprised of a network of natural and/or artificial fibers often referred to as threads or yarns. Yarn is produced by spinning raw wool fibers, linen, cotton, or other material on a spinning wheel to produce long strands known as yarn. Synthetic yarns are also available in the form of filament yarn. Textiles are formed by weaving, knitting, crocheting, knotting, or pressing fibers together. Textiles can be made from many materials. These materials come from four main sources: animal, plant, mineral, and synthetic. The fibre- or yarn-based materials may also consist of copolymers such as block-copolymers of olefins (e.g. ethylene/propylene) and styrene block-copolymers, and may comprise additives or fillers such as softeners and pigments. The fibre- or yarn-based materials may comprise a mixture of different fibres and the fibres may be of mono-, bi- or multicomponent type. The fibre- or yarn-based material may be elastic.

A fabric is a textile material. The word fabric is commonly used in textile assembly trades (such as tailoring and dressmaking) as a synonym for textile. However, there are subtle differences in these terms. Textile refers to any material made of interlacing fibres. Fabric refers to any material made through weaving, knitting, crocheting, or bonding. Generally, fabrics can be said to be fibre-based products having a substantial surface extent in relation to their thickness.

Nonwoven fabrics are those which are neither woven nor knit, for example felt. They are typically manufactured by putting staple fibers together in the form of a sheet or web, and then binding them either mechanically (as in the case of felt, by interlocking them with serrated needles so that the inter-fiber friction results in a stronger fabric), with an adhesive, or thermally (by applying binder (in the form of powder, paste, or polymer melt) and melting the binder onto the web by increasing temperature). Other manufacturing techniques involve the direct thermal bonding of meltspun fibres. Spunlaid nonwovens are made in one continuous process. Fibers are spun and then directly dispersed into a web by deflectors or can be directed with air streams. Several variants of this concept are available. Spunbond has been combined with meltblown nonwovens, co-forming them into a layered product called SMS (spunbond-meltblown-spunbond). Meltblown nonwovens have extremely fine fiber diameters but are not strong fabrics. Spunlaid is thermally bonded or bonded using a resin.

Foam can also be used as the ventilation material. An important characteristic of foams is their cell structure. To be useful in this invention, the foams need to have a high porosity and the majority of the cells needs to be of the open-celled type to allow sufficient air permeability. The foams may be reinforced by particles or fibre-shaped filler materials during manufacturing of the foams to achieve a higher compression resistance. The foams can be made of a variety of synthetic and natural polymers such as for example polyurethanes, polyethers, starch, cellulose and polystyrene and its derivatives. If necessary, the foam may be surface modified to achieve appropriate surface chemistry, for example be made hydrophobic.

Corrugated structures are also useful as the ventilation material. A corrugated structure may consist of a folded fibrous web material. The structures normally need to be stabilized to prevent the corrugations from pulling apart and flattening out. Examples of corrugated structures made from folded web materials, which may be utilized as the ventilation material, are described in EP 0 137 644. As described above, corrugated structures may also be formed by laminating a web or sheet material (such as a nonwoven or a film) on each side of a distance-giving material. If necessary, the outer layers may be additionally perforated in order to provide further enhanced air permeability and thereby comfort properties.

Alternatively, the ventilation material may be a sandwich material comprising two parallel layers between which a material composed of straw-like channels is positioned. The channels in the straw-like structure extend essentially in parallel with the two layers. The cross section of said straw like channels may have any suitable shape such as round, hexagonal, orthogonal. The walls of the channels may be apertured. The direction of said channel being parallel with the longitudinal direction or transversal direction allowing ventilation of the product.

Optionally, the ventilation material is hydrophobic, whereby it may be treated with an agent such as e.g. a surfactant in order to adjust the wettability thereof. Specific examples of materials that may be used as the ventilation material according to the invention are AirX™ (Tytex A/S, Ikast, Denmark), 3 mesh (Muller Textil GmbH, Wiehl, Germany), Scotknit 3D (Scott & Fyfe Ltd, Scotland) and Yuanfeng DT16-350 (Yuanfeng Textiles Co., Ltd, Fujian, China).

Furthermore, the first embodiment of the absorbent article 1 is gas permeable in such a way that passage of gas out of the article 1 via the ventilation components 14, 15 is allowed when the article is worn. More specifically, in the first embodiment each ventilation component 14, 15 has one associated gas passage opening. More specifically, the first ventilation component 14 has an associated first gas passage opening 16 formed in the front end edge 5 and the second ventilation component 15 has an associated second gas passage opening 17 formed in the rear end edge 6. The first and second gas passage openings 16, 17 constitute openings through which gas may flow out of, and into, the absorbent article 1. In the present application, the term “first gas passage opening” means a gas passage opening associated with a first ventilation component and the term “second gas passage opening” means a gas passage opening associated with a second ventilation component.

In the first embodiment, the first ventilation component 14 is positioned adjacent to the front end edge 5 and the second ventilation component 15 is positioned adjacent to the second end edge 6. More specifically, the first ventilation component 14 is positioned adjacent to the first gas passage opening 16 in the front end edge 5 and the second ventilation component 15 is positioned adjacent to the second gas passage opening 17 in the rear end edge 6. By the expression that “a ventilation component is positioned adjacent to a gas passage opening” is herein meant that a ventilation component is positioned at least partly at, or at least partly in, the gas passage opening such that the ventilation material is at least partly “open outwards” by means of the opening, whereby gas may pass directly from the ventilation material to the outside of the article. The first and second gas passage openings 16, 17 may be formed in the respective end edges 5, 6 by the fact that the backsheet 8 and topsheet 10 are not sealed to each other in the respective areas forming the respective gas passage openings 16, 17. FIG. 1b is a side view of the first embodiment shown in FIG. 1a, seen from the front end edge 5.

In the first embodiment, the first and second gas passage openings 16, 17 have essentially the same extension in the transverse (T) direction as the first and second ventilation components 14, 15, respectively. Furthermore, the first and second gas passage openings 16, 17 extend between essentially the same positions in the transverse (T) direction as the first and second ventilation components 14, 15, respectively.

As mentioned above, the combination of the mentioned thickness, porosity and ratio of the thickness at 0.5 kPa and 5 kPa of the ventilation material allows gas flow into, within and out of the ventilation components 14, 15. In addition, gas flow out of the article 1 is allowed by means of the respective gas passage openings 16, 17. Thereby, passage of gas out of the article 1 via the ventilation components 14, 15 and their associated gas passage openings 16, 17 is allowed, thus promoting ventilation of the absorbent article 1. FIG. 1c shows a cross-section of the first embodiment of the article 1 according to line I-I in FIG. 1a when the article 1 is worn by a wearer. The wearer is denoted by 20.

More specifically, as mentioned above, absorbent articles are usually in a particularly close contact with the wearer's skin in the area around the waist, which may imply that the transport of gas, i.e. ventilation, is inhibited there. The risk of development of skin irritations and rashes is usually particularly high in the area around the waist due to the close contact between the wearer's skin and the article in this area. By positioning the ventilation components 14, 15 in the first and second margin sections 11, 12, respectively, spacings between the backsheet 8 and the wearer's skin (or the topsheet 10) is created in the area around the waist. Gas flow is promoted into, within and out of the spacings due to the characteristics of the ventilation material making up the ventilation components 14, 15. Thus, the arrangement of the ventilation components 14, 15 imply that the passage of gas is improved at areas of the article 1 in which passage of gas usually is inhibited. Thereby, an improved circulation is achieved. In addition, gas flow is also allowed out of the article 1 by means of the respective gas passage openings 16, 17. By allowing gas flow into, within, and out of the ventilation components 14, 15 and out of the article 1 through the respective gas passage openings 16, 17, moist air, warm air, vapour and/or humidity may be transported away from the area between the wearer's skin and the topsheet 10 at the ventilation components 14, 15 and/or away from the ventilation components 14, 15. More specifically, moist air, warm air, vapour and/or humidity may be transported from the area between the article 1 and the wearer's skin through the topsheet 10 and out of the article 1 via the respective ventilation components 14, 15 and gas passage openings 16, 17. Thus, ventilation of the article 1 is promoted. Thereby, the temperature and the humidity level between the wearer's skin and the article 1 may be reduced, which implies that the risk of skin irritations and rashes is reduced.

However, the ventilation components 14, 15 and the gas passage openings 16, 17 imply not only that the transport of moist air, warm air, vapour and/or humidity is promoted away from the area between the article 1 and the wearer's skin at the ventilation components 14, 15, but also away from the area between the article 1 and the wearer's skin at other parts of the article as well as away from other parts within the article 1. Thereby, the temperature and humidity level between the wearer's skin and the article 1 may be reduced at all parts of the article, i.e. the complete microclimate may be improved. In addition, the temperature and the humidity level within the article 1 may be reduced. This implies that the risk of development of skin irritations and rashes is reduced and that the comfort is increased.

The transport of gas via the ventilation components may partly rely on diffusion. However, most importantly, ventilation in the form of convection is promoted in the first embodiment.

As may be realized from FIG. 1c, the first and second gas passage openings 16, 17 in the first embodiment imply that the article 1 is open “upwards” in the front and rear end edges 5, 6 when it is worn. Furthermore, each ventilation component 14, 15 serve as a channel, or conduit, for transportation of moist air, warm air, vapour and/or humidity (e.g. moisture from the area between the wearer's skin and the respective ventilation component 14, 15) to the respective gas passage opening 16, 17. Thereby a “chimney” effect, i.e. convection, may be achieved. More specifically, the “chimney” effect is promoted by means of the characteristics of the ventilation material, which imply that the ventilation components 14, 15 constitute channels for transport of gas and that the channels are maintained “open” for passage when the article 1 is worn, and by means of the gas passage openings 16, 17. Furthermore, by promoting ventilation in the form of convection a more efficient ventilation process, i.e. a more efficient process for transporting away moist air, warm air, vapour and/or humidity, is achieved compared to gas transport through diffusion only. This implies a reduced risk of development of skin irritations and/or rashes.

Furthermore, the ventilation that may be achieved according to the present invention may be active or passive as above defined. Natural or forced convection may be achieved. Forced convection may be achieved, for example, when the wearer sits down.

In addition, the absorbent article 1 shown in FIG. 1a comprises tape tabs 18 for fastening the absorbent article 1 on the wearer. Other absorbent articles according to the invention may comprise other types of fasteners such as friction fasteners, mechanical fasteners like hook-and-loop fasteners or adhesive fasteners for fastening the absorbent article to the underwear or around the waist of the wearer. Some absorbent articles are in the form of pants and therefore do not need special fastening means. In other cases the absorbent article is worn in special elastic pants without the need for additional fasteners.

Elastic elements (not shown) may be included in the absorbent article according to the invention to improve fit and reduce leakage. Exemplary elastic elements are waist elastics, leg elastics and so-called “standing gathers” (barrier flaps). Suitable elastic elements are known to the skilled person and need not be discussed further here.

The absorbent article shown in FIG. 1a has a generally I-shape. However, it may have any suitable shape. For example, a diaper according to the invention may have an overall rectangular shape, T-shape or an hour-glass shape.

In FIG. 1a the first and second ventilation components 14, 15 are illustrated as being rectangular in the L-T plane. However, they may in variations of the first embodiment have any other suitable shape in the L-T plane, such as a triangular shape, a circular shape or the shape of any other parallelogram. They may also have the shape of a funnel.

In another variant of the first embodiment, the first gas passage opening 16 and/or the second gas passage opening 17 do not extend between the same positions in the transverse (T) direction as the first ventilation component 14 and the second ventilation component 15, respectively, but extend between other positions. In still another variant of the first embodiment, the extension of the first gas passage opening 16 and/or the second gas passage opening 17 in the transverse (T) direction is different from (i.e. greater or smaller than) the extension of the first and second ventilation components 14, 15, respectively, in the transverse (T) direction. Furthermore, the first embodiment may also be varied such that each first ventilation component 14 has more than one associated first gas passage opening 16 in the front end edge 5 and/or such that each second ventilation component 15 has more than one associated second gas passage opening 17 in the rear end edge 6. In addition, the article 1 may comprise one or more first gas passage openings 16 in one of or both the side edges 7, i.e. in portions of the side edges 7 positioned in the front end section 2. Likewise, the article 1 may comprise one or more second gas passage openings 17 in one of or both the side edges 7, i.e. in portions of the side edges 7 positioned in the rear end section 3.

The article 1 may comprise any suitable number of first and second gas passage openings 16, 17. However, in the first embodiment, at least one first ventilation component 14 is positioned adjacent to at least one associated first gas passage opening 16 and/or at least one second ventilation component 15 is positioned adjacent to at least one associated second gas passage opening 17.

The first embodiment of the absorbent article 1 may also be varied in that the backsheet 8 further comprises a gas permeable panel 19 in the front end section 2 and a gas permeable panel 19 in the rear end section 3. FIG. 1d is a plan view of such a variation of the first embodiment, in which the absorbent article 1 is seen from the garment-facing side and in which the gas permeable panels 19 are positioned underlying the first and second ventilation components 14, 15, respectively.

Instead of being positioned completely underlying the respective ventilation components 14, 15, the respective gas permeable panels 19 may be positioned partly underlying the respective ventilation components 14, 15 or may be positioned in other parts of the backsheet 8. In addition, the backsheet 8 of the first embodiment may comprise any suitable number of (i.e. one or more) gas permeable panels 19. One or more of the gas permeable panels 19 may then be positioned at least partly underlying at least one ventilation component 14, 15 or may be positioned in other parts of the backsheet 8. However, each gas permeable panel 19 is preferably located in a margin section.

Each gas permeable panel 19 is formed as an integral portion of the backsheet 8 and is a panel through which gas may enter into the article 1 and flow out of the article 1. Each gas permeable panel 19 is substantially liquid impermeable or is positioned in areas where liquid normally does not reach. Each gas permeable panel 19 is constructed from a gas permeable material having an air permeability of at least 50 m³/m²/min, preferably at least 150 m³/m²/min, more preferably at least 300 m³/m²/min, most preferably at least 450 m³/m²/min as measured at 200 kPa and on a sample area of 20 cm² according to the method WSP 70.1 (05). The air permeability of the material of the gas permeable panels 19 is different from (i.e, higher than) the remainder of the backsheet 8.

Each gas permeable panel 19 may be formed from a material being different from the backsheet 8, whereby the gas permeable panel material may be joined to the remainder of the backsheet 8 by means of, for example, welding or gluing. Suitable materials for the gas permeable panels 19 may be nonwoven materials or perforated films. Nonwoven materials can be spunlaid, meltblown, carded, air-laid, wet-laid or multi-layered combinations thereof, such as SMMS (spunlaid-meltblown-meltblown-spunlaid). The nonwoven materials may be bonded using any conventional techniques well-known to those skilled in the art for example by thermo-bonding (hot air or point-embossing), latex bonding and mechanical bonding such as hydroentangling. The fibres may be of natural or synthetic origin such as cotton, viscose, rayon, polyesters, polyurethanes, polyamides, polypropylene, polyethylene and polyacrylates. The fibres may also consist of various kinds of copolymers such as block-copolymers of olefins (e.g. ethylene/propylene) and styrene block-copolymers and may comprise various kind of additives or fillers such as titaniumdioxide, etc. The nonwovens may consist of a mixture of different fibres and the fibres may be of mono-, bi- or multicomponent type. The nonwoven may be elastic. If required, the nonwoven material may be apertured.

Perforated plastic films may also be used as a material for the gas permeable panels 19. The plastic film can be made of any suitable polymer although the most common ones would be polyethylene and polypropylene. The films may contain additives and/or fillers such as pigments and calcium carbonate. The number and size of perforations need to be set in relation to the required air permeability and material strength.

The material of the gas permeable panels 19 is preferably hydrophobic to further enhance the material's barrier properties against liquids.

The material of the gas permeable panel 19 may be, for example, 30 gsm PP SMMS (WHITES107007, Fibertex A/S), 17 gsm PP SSS, 1.1-1.3 dtex (Elite Soft, Fibertex A/S), 30 gsm perforated biodegradable film (PVFX 271-P9, Poligof), 36 gsm perforated PE film (Aquidry Classic, Tredegar) and 15 gsm PP (4W H05-01; Fiberweb).

Alternatively, a gas permeable panel 19 may be formed by the fact that the backsheet 8 is perforated in the area forming the gas permeable panel 19, whereby the backsheet 8 is perforated so as to achieve the above mentioned air permeability. Furthermore, in case the backsheet 8 consists of a laminate of a nonwoven material and a plastic film, the plastic film may be removed in a certain area so as to form a gas permeable panel 19 in that area of the backsheet 8. The nonwoven material has then the above mentioned air permeability in the gas permeable panel 19.

By means of the gas permeable panels 19, ventilation of the article 1 may be further promoted. Gas, e.g. air, from outside of the article 1 may enter into the article 1, and thus into the ventilation components 14, 15, through the respective gas permeable panels 19. Increased gas flow into the article 1 implies that gas within the article 1 is more efficiently replaced, whereby transport of gas out of the article 1 via the ventilation components 14, and the respective associated gas passage openings 16, 17 is more efficient. Thereby, ventilation of the article 1 is further promoted. In particular, ventilation in the form of convection is further promoted. Consequently, the temperature and the humidity level between the wearer's skin and the topsheet 10, as well as within the article 1, may be more efficiently reduced, which implies that the risk of development of skin irritations and rashes is more efficiently reduced.

In order to promote ventilation through convection, the minimum dimension of each gas permeable panel 19 in any direction in the L-T plane may be, for example, at least half the thickness of any of the ventilation components 14, 15 and preferably at least the same dimension as the thickness of the ventilation components 14, 15. The total area of all gas permeable panels 19 formed in the backsheet 8 may be, for example, up to two thirds of the area of the backsheet 8.

In FIG. 1d the gas permeable panels 19 are illustrated as being rectangular in the L-T plane. However, they may in variations of the first embodiment have any other suitable shape in the L-T plane, such as triangular, circular or the shape of any other parallelogram.

Furthermore, the first embodiment may also be varied (not shown) such that it comprises more than one first ventilation component 14 in the first margin section 11 and/or more than one second ventilation component 15 in the second margin section 12. In case the article 1 comprises more than one first ventilation component 14, it may comprise one or more first gas passage openings 16 in the front end edge 5, i.e. each first ventilation component 14 has at least one associated first gas passage opening 16 in the front end edge 5. Likewise, in case the article 1 comprises more than one second ventilation component 15, it may comprise one or more second gas passage openings 17 in the rear end edge 6, i.e. each second ventilation component 15 has at least one associated second gas passage opening 17 in the rear end edge 6.

In another variant (not shown) of the first embodiment, the article 1 according to the invention comprises one or more first ventilation components 14 in the first margin section 11 and thereto associated first gas passage opening(s) 16 in the front end edge 5, but no second ventilation components 15 in the second margin section 12 and no second gas passage openings 17 in the rear end edge 6. Likewise, in a further variant (not shown) of the first embodiment, the article 1 according to the invention comprises one or more second ventilation components 15 in the second margin section 12 and thereto associated second gas passage opening(s) 17 in the rear end edge 6, but no first ventilation components 14 in the first margin section 11 and no first gas passage openings 16 in the front end edge 5.

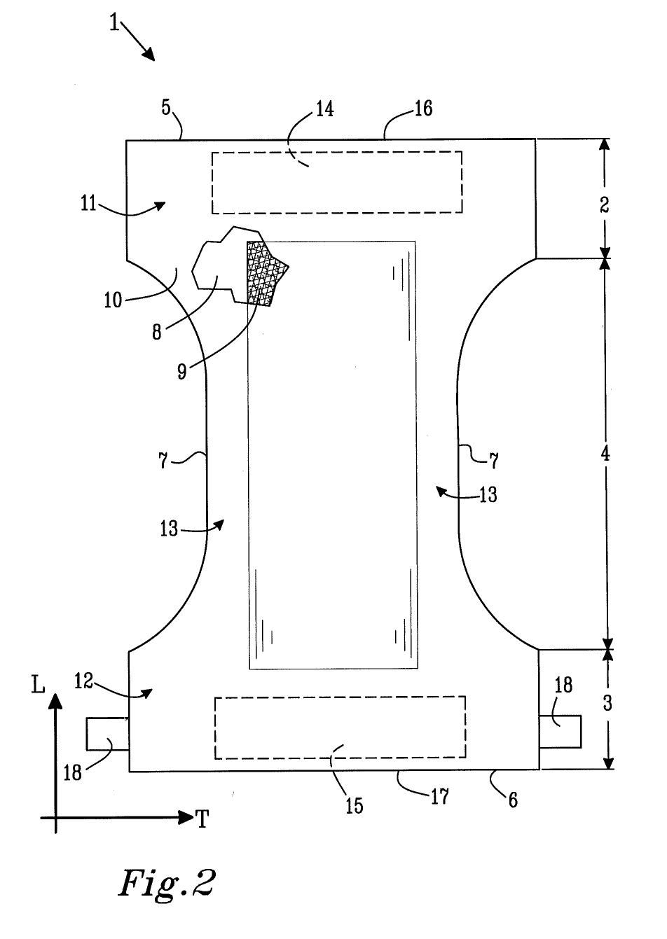

FIG. 2 is a plan view of a second embodiment of an absorbent article 1 according to the invention, in this case a diaper, seen from the wearer-facing side. The second embodiment corresponds to the first embodiment shown in FIG. 1a except for the fact that the first and second ventilation components 14, 15 are positioned spaced from the front and rear end edges 5, 6, respectively, i.e. they do not extend to the front and rear end edges 5, 6, respectively, whereby there is a certain distance between the first and second ventilation components 14, 15 and the front and rear end edges 5, 6, respectively. Thus, the ventilation components 14, 15 are thereby also positioned spaced from the associated first and second gas passage openings 16, 17. The expression “spaced from” is herein intended to mean that a ventilation component is positioned at a certain distance, for example 3-20 mm, from another component (e.g. an edge or a gas passage opening) in any direction in the L-T plane. However, the ventilation components 14, 15 are still positioned in the first and second margin sections 11, 12, respectively. In the second embodiment gas flow into, within and out of the ventilation components 14, 15 and out of the article 1 through the first and second gas passage openings 16, 17 is promoted in the same way as in the first embodiment. Furthermore, the second embodiment may be varied in accordance with the above described variations of the first embodiment. In case the second embodiment comprises more than one first ventilation component 14, at least one first ventilation component 14 is spaced from its associated first gas passage opening(s) 16. Likewise, in case the second embodiment comprises more than one second ventilation component 15, at least one second ventilation component 15 is positioned spaced from its associated second gas passage opening(s) 17.

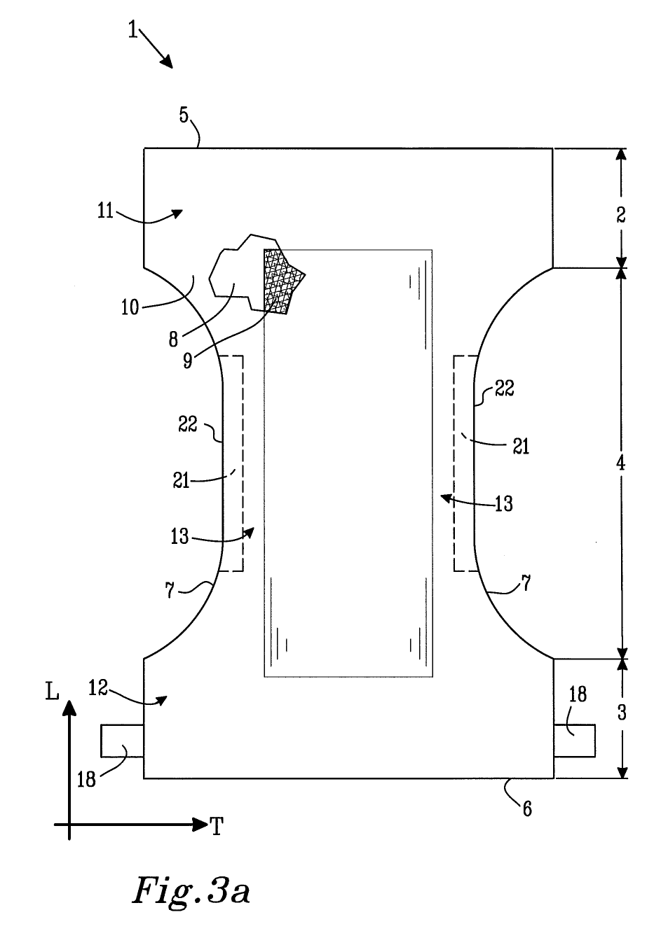

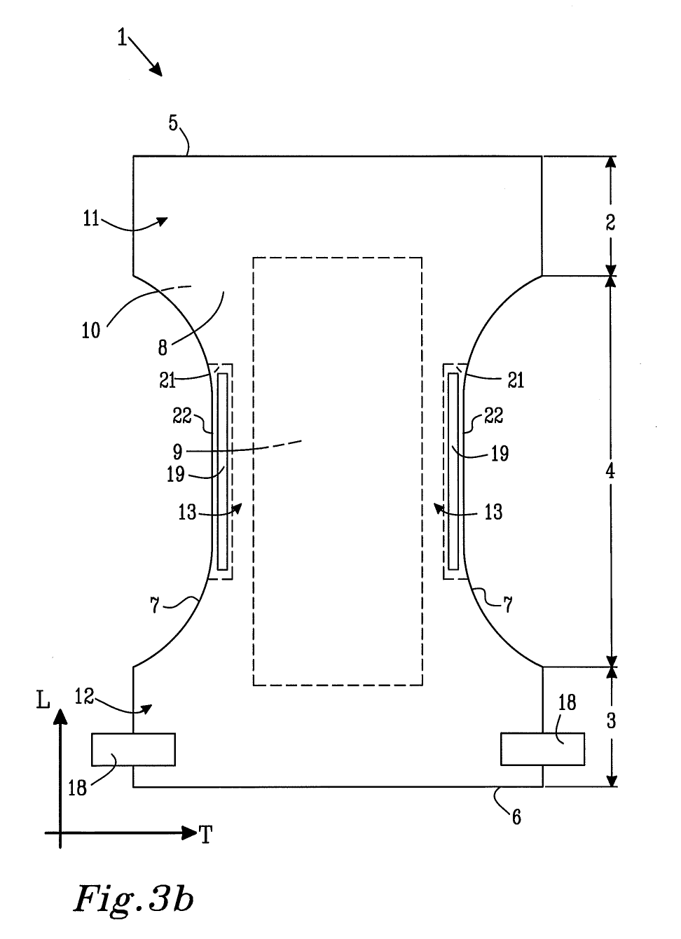

FIG. 3a is a plan view of a third embodiment of an absorbent article 1 according to the invention, in this case a diaper, seen from the wearer-facing side. The third embodiment corresponds to the first embodiment except for the fact that it does not comprise any first or second ventilation components 14, 15. Furthermore, the third embodiment does not comprise any first or second gas passage openings 16, 17. Instead, the third embodiment comprises one third ventilation component 21 in each third margin section 13. As mentioned above, the absorbent article 1 comprises one third margin section 13 on each side of the absorbent core 9 in the crotch section 4. Each third margin section 13 is constituted by the portion of the absorbent article 1 at one side edge 7 in the crotch section 4 in which the backsheet 8 is not overlaid by the absorbent core 9. Each third margin section 13 is positioned adjacent to one of the side edges 7 in the crotch portion 4. Each third ventilation component 21 is constituted by the ventilation material described above for the first and second ventilation components 14, 15 in the first embodiment. Each third ventilation component 21 is positioned overlaying the backsheet 8. More specifically, each third ventilation component 21 is positioned between the backsheet 8 and the topsheet 10 in the third embodiment. As may be seen in FIG. 3a, the third ventilation components 21 are arranged such that they are exclusively localized in the respective third margin sections 13 in the third embodiment. However, alternatively one or both of the third ventilation components 21 may be arranged such that it is partly localized in one third margin section 13 and partly in a first margin section 11 and/or a second margin section 12. This will be further described below. In the present application, the term “third ventilation component” means a ventilation component that is at least partly localized in one third margin section. However, the third ventilation components 21 do not overlap with, or extend over, the absorbent core 9, i.e. the ventilation material is only found in the third ventilation components 21 and does not extend over the absorbent core 9.

Furthermore, the absorbent article 1 is gas permeable in such a way that passage of gas out of the article 1 via the third ventilation components 21 is allowed when the article is worn. In the third embodiment, each third ventilation component 21 has an associated third gas passage opening 22 formed in the respective side edge 7 adjacent to the respective third margin section 13 in which the respective third ventilation component 21 is comprised. In the present application, the term “third gas passage opening” means a gas passage opening associated with a third ventilation component.

In the third embodiment, each third ventilation component 21 is positioned adjacent to the respective side edge 7. More specifically, each third ventilation component 21 is positioned adjacent to the respective associated third gas passage opening 22.

The third ventilation components 21 may be attached to the backsheet 8 and/or topsheet 10 in one or more regions by any known method in the art, such as by means of adhesive, heat bonding, etc. Furthermore, the third gas passage openings 22 may be formed in the respective side edges 7 by the fact that the backsheet 8 and topsheet 10 are not sealed to each other in the respective areas forming the respective gas passage openings 22.

In the third embodiment, the third gas passage openings 22 have essentially the same extension in the longitudinal (L) direction as the third ventilation components 21, respectively. Furthermore, the third gas passage openings 22 extend between essentially the same positions in the longitudinal (L) direction as the third ventilation components 21.

The combination of the mentioned thickness, porosity and ratio of the thickness at 0.5 kPa and 5 kPa of the ventilation material allows gas flow into, within and out of the third ventilation components 21. In addition, gas flow out of the article 1 is allowed by means of the respective third gas passage openings 22. Thereby, passage of gas out of the article 1 via the ventilation components 21 and their associated third gas passage openings 22 is allowed, thus promoting ventilation of the absorbent article 1.

More specifically, as mentioned above, absorbent articles are usually in a particularly close contact with the wearer's skin in the area at the legs (e.g. at leg openings), which may imply that the transport of gas, i.e. ventilation, is inhibited there. The risk of development of skin irritations and rashes is usually particularly high in the area at the legs due to the close contact between the wearer's skin and the article in this area. By positioning the third ventilation components 21 in the respective third margin sections 22, spacings between the backsheet 8 and the wearer's skin (or the topsheet 10) is created in the area at the legs. Gas flow is promoted into, within and out of the spacings due to the characteristics of the ventilation material making up the third ventilation components 21. Thus, the arrangement of the third ventilation components 21 imply that the passage of gas is improved at areas of the article 1 in which passage of gas usually is inhibited. Thereby, an improved circulation is achieved. In addition, gas flow is also allowed out of the article 1 by means of the respective gas passage openings 22. By allowing gas flow into, within, and out of the ventilation components 21 and out of the article 1 through the respective gas passage openings 22, moist air, warm air, vapour and/or humidity may be transported away from the area between the wearer's skin and the topsheet 10 at the ventilation components 21 and/or away from the ventilation components 21. More specifically, moist air, warm air, vapour and/or humidity may be transported from the area between the article 1 and the wearer's skin through the topsheet 10 and out of the article 1 via the respective ventilation components 21 and gas passage openings 22. Thus, ventilation of the article 1 is promoted. Thereby, the temperature and the humidity level between the wearer's skin and the article 1 may be reduced, which implies that the risk of skin irritations and rashes is reduced.

However, the ventilation components 21 and the gas passage openings 22 imply not only that the transport of moist air, warm air, vapour and/or humidity is promoted away from the area between the article 1 and the wearer's skin at the ventilation components 21 due to the spacing of the article 1 from the wearer, but also away from the area between the article 1 and the wearer's skin at other parts of the article as well as away from other parts within the article 1. Thereby, the temperature and humidity level between the wearer's skin and the article 1 may be reduced at all parts of the article, i.e. the complete microclimate may be improved. In addition, the temperature and the humidity level within the article 1 may be reduced. This implies that the risk of development of skin irritations and rashes is reduced and the comfort is increased.

The transport of gas via the ventilation components may partly rely on diffusion. However, most importantly, ventilation in the form of convection is promoted in the third embodiment. Furthermore, the ventilation that may be achieved according to the present invention may be active or passive as above defined. Natural or forced convection may be achieved, Forced convection may be achieved, for example, when the wearer sits down.

In FIG. 3a the third ventilation components 21 are illustrated as being rectangular in the L-T plane. However, they may in variations of the third embodiment have any other suitable shape in the L-T plane, such as a triangular shape, a circular shape, a wave shape, a moon shape or the shape of any other parallelogram.

In another variant of the third embodiment, the third gas passage openings 22 do not extend between the same positions in the longitudinal (L) direction as the third ventilation components 21, but extend between other positions. In still another variant of the third embodiment, the extension of the third gas passage openings 22 in the longitudinal (L) direction is different from (i.e. greater or smaller than) the extension of the third ventilation components 21 in the longitudinal (L) direction. Furthermore, the third embodiment may also be varied such that each third ventilation component 21 has more than one associated third gas passage opening 22 in the respective side edge 7.

The article 1 may comprise any suitable number of third gas passage openings 22 in each side edge 7. However, in the third embodiment, at least one third ventilation component 21 is positioned adjacent to at least one third gas passage opening 22 associated therewith.

Furthermore, the extension of each of the third components 21 in the longitudinal (L) and transverse (T) directions, respectively, of the absorbent article 1 depends on the size of the article 1, i.e. it depends on the type of the article 1. Thus, the extension of the third ventilation components 21 may differ between e.g. incontinence products for adults and diapers for premature babies. For example, each third ventilation component 21 may extend about 5-20 mm in the transverse (T) direction of the article 1, i.e. each third ventilation component 21 may have a maximum extension in the transverse (T) direction of about 5-20 mm. The extension of the respective third ventilation components 21 in the longitudinal (L) direction of the absorbent article 1 may be, for example, from 1 cm and up to the length of the absorbent article 1 in the longitudinal (L) direction. However, concerning dimensions of the third ventilation components 21, the thickness of the ventilation components 21 is the most important characteristic in order to achieve the inventive concept.

The third embodiment of the absorbent article 1 may also be varied in that the backsheet 8 further comprises one or more gas permeable panels 19. FIG. 3b is a plan view of such a variation of the third embodiment, in which the absorbent article 1 is seen from the garment-facing side and in which one gas permeable panel 19 is positioned underlying each of the third ventilation components 21. The gas permeable panels 19 in the third embodiment correspond to the gas permeable panel 19 described in the first embodiment. As described above, by means of the gas permeable panels 19, ventilation of the article 1 may be further promoted.

Instead of being positioned completely underlying the respective ventilation components 21, the respective gas permeable panels 19 may be positioned partly underlying the respective ventilation components 21 or may be positioned in other parts of the backsheet 8. In addition, the backsheet 8 of the third embodiment may comprise any suitable number of gas permeable panels 19 (i.e. one or more gas permeable panels 19). One or more of the gas permeable panels 19 may then be positioned at least partly underlying at least one ventilation component 21 or may be positioned in other parts of the backsheet 8. However, each gas permeable panel 19 is preferably located in a margin section.

In order to promote ventilation through convection, the minimum dimension of each gas permeable panel 19 in any direction in the L-T plane may be, for example, at least half the thickness of any of the ventilation components 21 and preferably the same dimension as the thickness of the ventilation components 21. The total area of all gas permeable panels 19 formed in the backsheet 8 may be, for example, up to two thirds of the area of the backsheet 8.

Furthermore, the third embodiment may also be varied (not shown) such that it comprises more than one third ventilation component 21 on each side of the absorbent core 9. Then the article 1 may comprise one or more third gas passage openings 22 in each side edge 7, i.e. each third ventilation component 21 may have at least one associated third gas passage opening 22.

In a further variation (not shown) of the third embodiment, at least one third ventilation component 21 is localized partly in one third margin section 13 and partly in at least one of a first margin section 11 in the front end section 2 and a second margin section 12 in the rear end section 3 such that it extends from the third margin section 13 into at least one of the first and second margin sections 11, 12.

In still a further variation (not shown) of the third embodiment, at least one third ventilation component 21 is localized partly in one third margin section 13, partly in a first margin section 11 in the front end section 2 and partly in a second margin section 12 in the rear end section 3 such that it extends from a first gas passage opening 16 formed in the front end edge 5 to a second gas passage opening 17 formed in the rear end edge 6.

In still another variation (not shown) of the third embodiment, the article 1 comprises standing gathers, whereby at least one third ventilation component 21 on each side of the absorbent core 9 is part of the standing gathers.

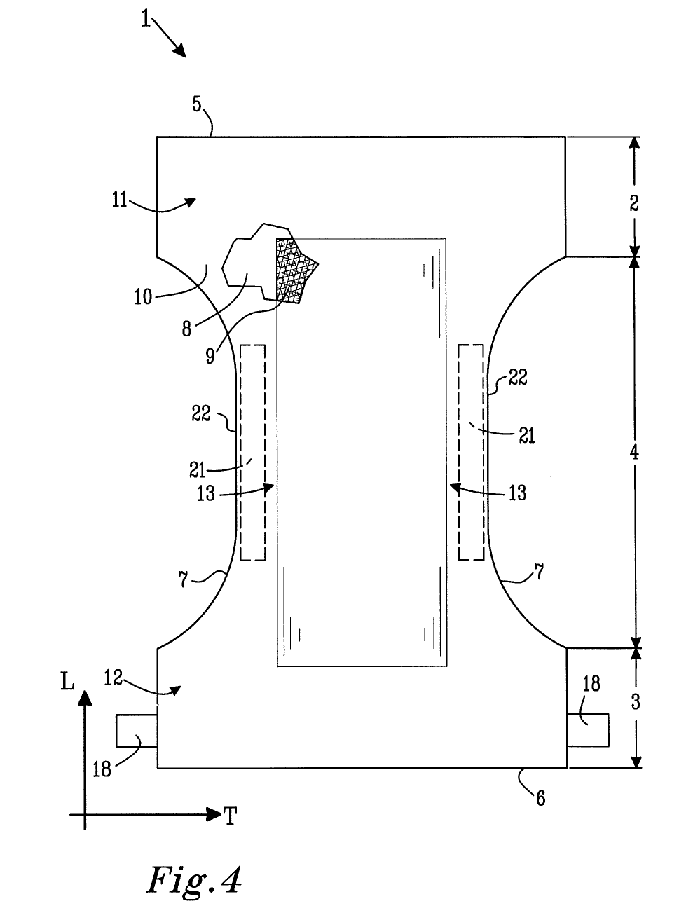

FIG. 4 is a plan view of a fourth embodiment of an absorbent article 1 according to the invention, in this case a diaper, seen from the wearer-facing side. The fourth embodiment corresponds to the third embodiment shown in FIG. 3a except for the fact that the third ventilation components 21 are positioned spaced from the respective side edges 7, i.e. they do not extend to the respective side edges 7, whereby there is a certain distance between the respective ventilation components 21 and the respective side edges 7. Thus, the third ventilation components 21 are thereby also positioned spaced from the third gas passage openings 22. In the fourth embodiment gas flow into, within and out of the ventilation components 21 and out of the article 1 through the third gas passage openings 22 is promoted in the same way as in the third embodiment. Furthermore, the fourth embodiment may be varied in accordance with the above described variations of the third embodiment. In case the fourth embodiment comprises more than one third ventilation component 21, at least one third ventilation component 21 may be positioned spaced from its associated third gas passage opening(s) 22.

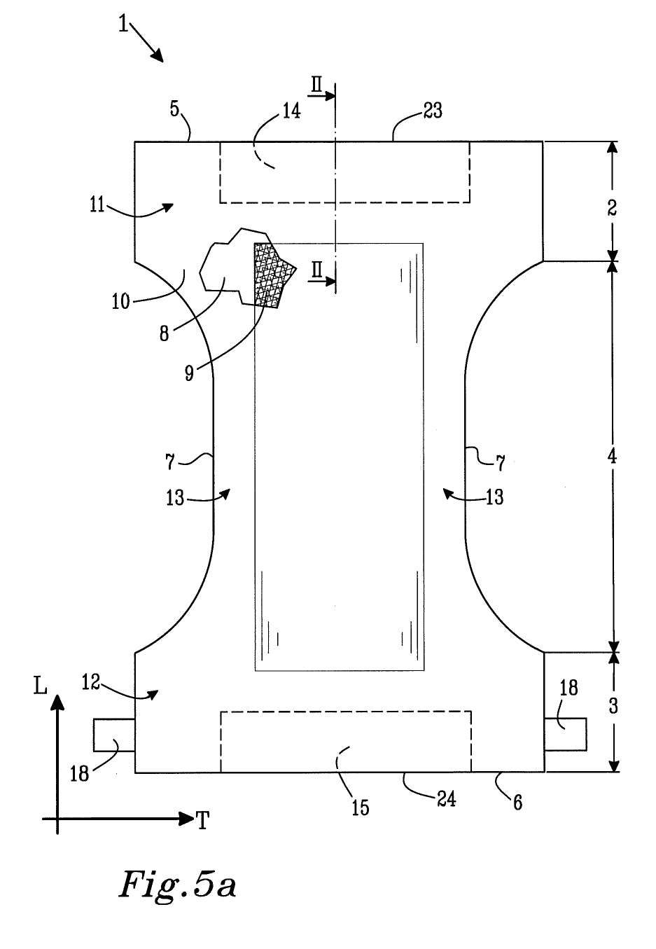

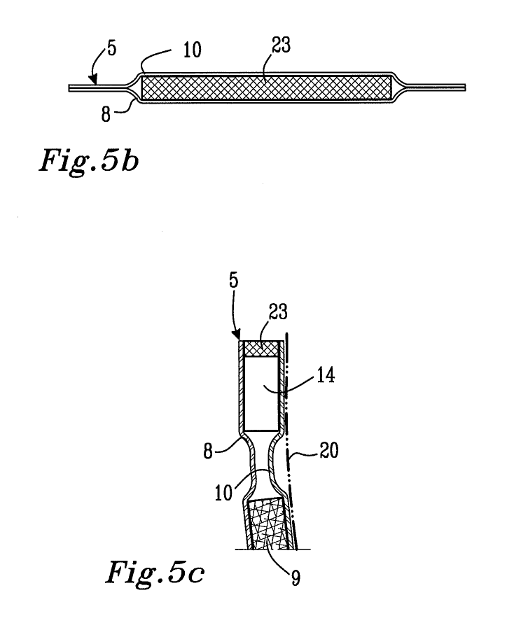

FIG. 5a is a plan view of a fifth embodiment of an absorbent article according to the invention, seen from the wearer-facing side. The fifth embodiment corresponds to the first embodiment shown in FIG. 1a except for concerning the fact that it does not comprise any first gas passage openings or second gas passage openings. Instead the fifth embodiment of the absorbent article 1 is gas permeable such that passage of gas out of the article 1 via the ventilation components 14, 15 is allowed when the article 1 is worn in that each of the first and second ventilation components 14, 15 has an associated gas permeable window formed in the front and rear end edge 2, 3, respectively. More specifically, in the fifth embodiment the first ventilation component 14 has an associated first gas permeable window 23 formed in the front end edge 5 and the second ventilation component 15 has an associated second gas permeable window 24 formed in the rear end edge 6. In the present application, the term “first gas permeable window” means a gas permeable window associated with a first ventilation component and the term “second gas permeable window” means a gas permeable window associated with a second ventilation component. The first gas permeable window 23 is formed by the fact that the topsheet 10 and the backsheet 8 are non-sealed in an area of the front end edge 5 and by the fact that a gas permeable material is arranged between the end of the topsheet 10 and the end of the backsheet 8 in the non-sealed area in the front end edge 5, i.e. the gas permeable material is joined to the end of the topsheet 10 and the end of the backsheet 8 such that it is a part of the front end edge 5. This may be seen in FIG. 5b, which is a side view of the fifth embodiment shown in FIG. 5a, seen from the front end edge 5. Likewise, the second gas permeable window 24 is formed by the fact that the topsheet 10 and the backsheet 8 are non-sealed in an area of the rear end edge 6 and by the fact that a gas permeable material is arranged between the end of the topsheet 10 and the end of the backsheet 8 in the non-sealed area in the rear end edge 6, i.e. the gas permeable material is joined to the end of the topsheet 10 and the end of the backsheet 8 such that it is a part of the rear end edge 6. The gas permeable material may, for example, be joined to the backsheet 8 and topsheet 10 by means of gluing or welding.

In the fifth embodiment, the first ventilation component 14 is preferably positioned adjacent to its associated first gas permeable window 23 such that the first gas permeable window 23 covers at least parts of the first ventilation component 14 at the front end edge 5. Likewise, the second ventilation component 15 is preferably positioned adjacent to its associated second gas permeable window 24 such that the second gas permeable window 24 covers at least parts of the second ventilation component 15 at the rear end edge 6. Each gas permeable window 23, 24 is a window through which gas may enter into the article 1 and flow out of the article 1.

The material of the gas permeable windows 23, 24 may be the material described above for the gas permeable panel 19. The gas permeable material of the gas permeable windows 23, 24 has an air permeability of at least 50 m³/m²/min, preferably at least 150 m³/m²/min, more preferably at least 300 m³/m²/min, most preferably at least 450 m³/m²/min as measured at 200 kPa and on a sample area of 20 cm² according to the method WSP 70.1 (05). In the fifth embodiment gas flow into, within and out of the ventilation components 14, 15 and out of the article 1 through the first and second gas permeable windows 23, 24 is allowed, thus promoting ventilation of the article 1 based on the same principle as in the first embodiment (i.e. the gas permeable windows 23, 24 take the place of the openings 16, 17 in the first embodiment). FIG. 5c shows a cross-section of the fifth embodiment according to line II-II in FIG. 5a when the article is worn by a wearer.

Furthermore, the fifth embodiment may be varied in accordance with the above described variations of the first embodiment. Thus, it may comprise more than one first ventilation component 14, whereby it may comprise more than one first gas permeable window 23 in the front end edge 5, i.e. each first ventilation component 14 has an associated first gas permeable window 23 in the front end edge 5. Likewise, it may comprise more than one second ventilation component 15, whereby it may comprise more than one second gas permeable window 24 in the rear end edge 6, i.e. each second ventilation component 15 has an associated second gas permeable window 24 in the rear end edge 6. Furthermore, the fifth embodiment may comprise one or more gas permeable panels 19 in the backsheet 8. In addition, the fifth embodiment may comprise one or more first ventilation components 14 and one or more first gas permeable windows 23, but no second ventilation components 15 and thereto associated second gas permeable windows 24, and vice versa.

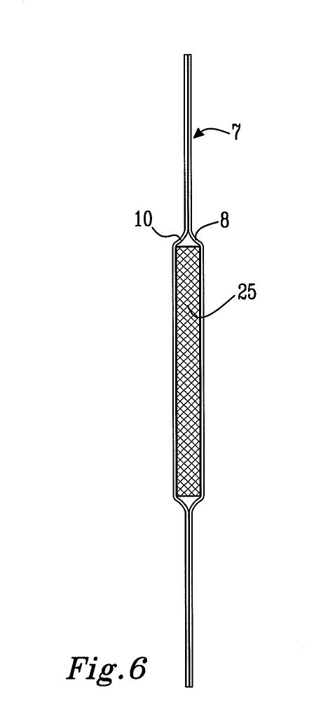

A sixth embodiment of the absorbent article 1 according to the invention corresponds to the third embodiment shown in FIG. 3a except for concerning the fact that it does not comprise any third gas passage openings. Instead the sixth embodiment of the absorbent article 1 is gas permeable such that passage of gas out of the article 1 via the third ventilation components 21 is allowed when the article 1 is worn in that each of the third ventilation components 21 has an associated gas permeable window formed in the respective side edges 7. More specifically, each third ventilation component 21 has one associated third gas permeable window 25 formed in the respective side edge 7 located adjacent to the respective third margin section 13 in which the respective third ventilation component 21 is localized. In the present application, the term “third gas permeable window” means a gas permeable window associated with a third ventilation component. Each third gas permeable window 25 is formed by the fact that the topsheet 10 and the backsheet 8 are non-sealed in an area of the respective side edge 7 and by the fact that a gas permeable material is arranged between the end of the topsheet 10 and the end of the backsheet 8 in the non-sealed area in the respective side edge 7, i.e. the gas permeable material is joined to the end of the topsheet 10 and the end of the backsheet 8 such that it is a part of the respective side edge 7. This may be seen in FIG. 6, which is a side view of the sixth embodiment seen from one of the side edges 7. The gas permeable material may, for example, be joined to the backsheet 8 and topsheet 10 by means of gluing or welding.

In the sixth embodiment, each third ventilation component 21 is preferably positioned adjacent to its associated third gas permeable window 25 such that the third gas permeable window 25 covers at least parts of the respective third ventilation component 21 at the respective side edge 7, i.e. each third ventilation component 21 has an associated gas permeable window 25 in a side edge 7. Each gas permeable window 25 is a window through which gas may enter into the article 1 and flow out of the article 1.

The material of the third gas permeable windows 25 correspond to the material of the gas permeable windows 23, 24 in the fifth embodiment. In the sixth embodiment gas flow into, within and out of the third ventilation components 21 and out of the article 1 through the third gas permeable windows 25 is allowed, thus promoting ventilation of the article 1 based on the same principle as in the third embodiment.

Furthermore, the sixth embodiment may be varied in accordance with the above described variations of the third embodiment. Thus, it may comprise more than one third ventilation component 21 in each third margin section 13, whereby it may comprise more than one third gas permeable window 25 in each side edge 7. Furthermore, the sixth embodiment may comprise one or more gas permeable panels 19 in the backsheet 8.

In one variation (not shown) of the sixth embodiment, at least one third ventilation component 21 is localized partly in one third margin section 13 and partly in at least one of a first margin section 11 in the front end section 2 and a second margin section 12 in the rear end section 3 such that it extends from the third margin section 13 into at least one of the first and second margin sections 11, 12.

In a further variation (not shown) of the sixth embodiment, at least one third ventilation component 21 is localized partly in one third margin section 13, partly in a first margin section 11 in the front end section 2 and partly in a second margin section 12 in the rear end section 3 such that it extends from a first gas permeable window 23 in the front end edge 5 to a second gas permeable window 24 in the rear end edge 6.

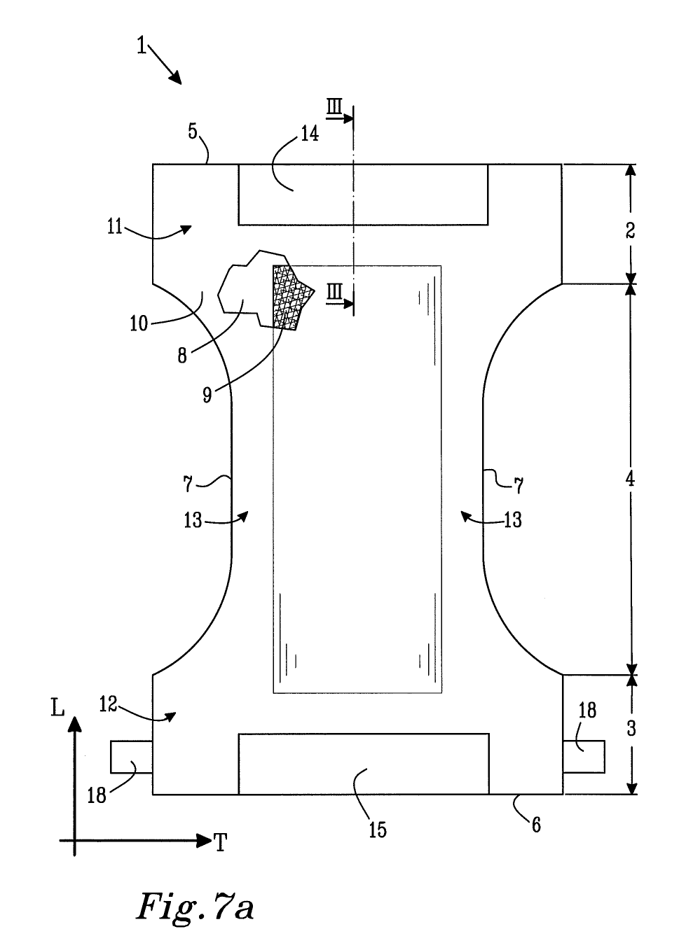

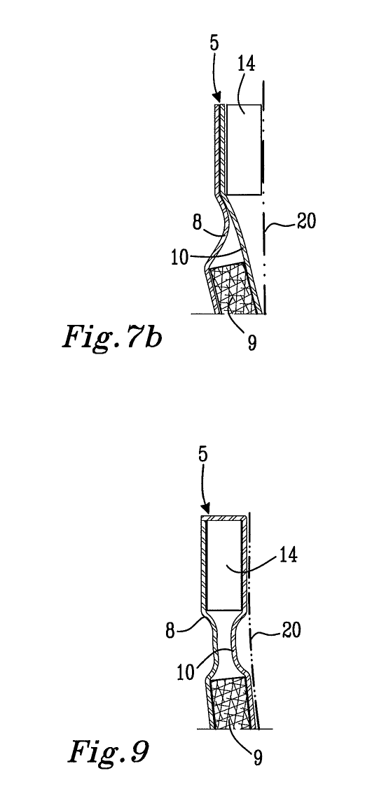

FIG. 7a is a plan view of a seventh embodiment of an absorbent article according to the invention, seen from the wearer-facing side. The seventh embodiment of the article 1 corresponds to the first embodiment except for concerning the fact that the first and second ventilation components 14, 15 are positioned overlaying, i.e. on, said topsheet 10 so as to be in contact with the wearer's skin during use of the article 1 and the fact that the seventh embodiment does not comprise any gas passage openings. Furthermore, in the seventh embodiment the first and second ventilation components 14, 15 may be comprised anywhere within the first and second margin sections 11, 12, respectively. This embodiment is gas permeable in such a way that passage of gas out of the article 1 via the ventilation components 14, 15 is allowed, i.e. gas may pass from the area between the article 1 and the wearer's skin (i.e. the microclimate) and out of the article 1 via the ventilation components 14, 15. Thus, the fact that the ventilation components 14, 15 are positioned on the topsheet 10 implies that gas flow between the article 1 and the wearer's skin is promoted, thus promoting ventilation between the article 1 and the wearer's skin.

Since the ventilation components 14, 15 are localized on the topsheet 10, i.e. on the outside of the article 1, gas may exit the ventilation components 14, 15 to the outside of the article 1 through any side of the ventilation components 14, 15 not being in contact with the wearer's skin or the topsheet 10 (i.e. through any side being in contact with the surroundings of the article 1). FIG. 7b shows a cross-section of the seventh embodiment according to line in FIG. 7a when the article is worn by a wearer. The seventh embodiment may be varied in accordance with the variations of the first embodiment. Thus, it may comprise more than one first ventilation component 14 and/or more than one second ventilation component 15. In addition, it may comprise one or more first ventilation components 14, but no second ventilation components 15. Alternatively, it may comprise one or more second ventilation components 15, but no first ventilation components 14. Furthermore, the seventh embodiment may comprise one or more gas permeable panels 19 in the backsheet 8. In case it comprises one or more gas permeable panels 19, gas may pass through the topsheet 10 into the ventilation components 14, 15 and via the ventilation components 14, 15 to the outside of the article 1.

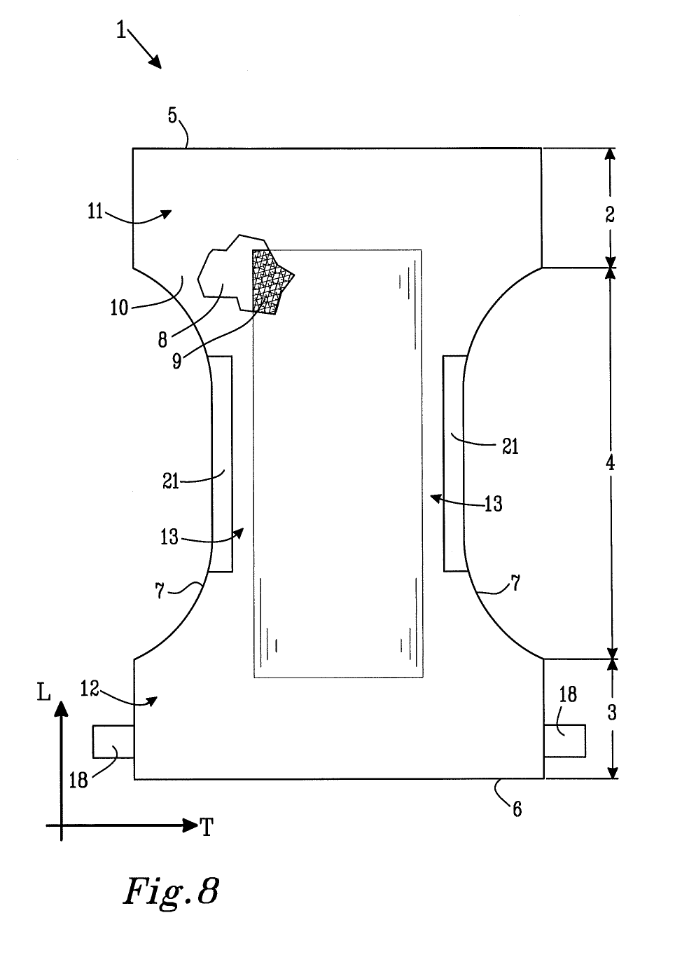

FIG. 8 is a plan view of an eighth embodiment of an absorbent article according to the invention, seen from the wearer-facing side. The eighth embodiment of the article 1 corresponds to the third embodiment except for concerning the fact that the third ventilation components 21 are positioned overlaying, i.e. on, said topsheet 10 so as to be in contact with the wearer's skin during use of the article 1 and the fact that the eighth embodiment does not comprise any gas passage openings. Furthermore, in the eighth embodiment the third ventilation components 21 are localized anywhere within the respective third margin sections 13. This embodiment is gas permeable in such a way that passage of gas out of the article 1 via the third ventilation components 21 is allowed, i.e. gas may pass from the area between the article 1 and the wearer's skin (i.e. the microclimate) out of the article 1 via the ventilation components 21. Thus, the fact that the third ventilation components 21 are positioned on the topsheet 10 implies that gas flow between the article 1 and the wearer's skin is promoted, thus promoting ventilation between the article 1 and the wearer's skin. Since the ventilation components 21 are localized on the topsheet 10, i.e. on the outside of the article 1, gas may exit the ventilation components 21 to the outside of the article 1 through any side of the ventilation components 21 not being in contact with the wearer's skin or the topsheet 10. Furthermore, the eighth embodiment may be varied in accordance with the above described variations of the third embodiment. Thus, it may comprise more than one third ventilation component 21 in each third margin section 13. In addition, the eighth embodiment may comprise one or more gas permeable panels 19 in the backsheet 8. In case it comprises one or more gas permeable panels 19, gas may pass through the topsheet 10 into the third ventilation components 21 and via the third ventilation components 21 to the outside of the article 1.

In one variation (not shown) of the eighth embodiment, at least one third ventilation component 21 is localized partly in one third margin section 13 and partly in at least one of a first margin section 11 in the front end section 2 and a second margin section 12 in the rear end section 3 such that it extends from the third margin section 13 into at least one of the first and second margin sections 11, 12.

In a further variation (not shown) of the eighth embodiment, at least one third ventilation component 21 is localized partly in one third margin section 13, partly in a first margin section 11 in the front end section 2 and partly in a second margin section 12 in the rear end section 3 such that it extends from the front end edge 5 to the rear end edge 6.

A ninth embodiment of the absorbent article 1 according to the invention corresponds to the first embodiment shown in FIG. 1a except for concerning the fact that it does not comprise any first gas passage openings or second gas passage openings 16, 17. Instead the fifth embodiment of the absorbent article 1 is gas permeable in such a way such that passage of gas out of the article 1 via the ventilation components 14, 15 is allowed when the article 1 is worn in that the topsheet 10 has such an extension that it at least partly extends over the first and second ventilation components 14, 15 in the Z direction of the article at the front and rear end edge 2, 3, respectively. Thus, the first and second ventilation components 14, 15 are at least partly covered at the front and rear end edge 5, 6, respectively by the topsheet 10. FIG. 9 is a cross-section of the ninth embodiment in use. The cross-section in FIG. 9 is taken in a similar way in the ninth embodiment as the cross-section in FIG. 1c taken in the first embodiment.

In the ninth embodiment, gas may pass into, within and out of the ventilation components 14, 15 and out of the article 1 through the topsheet 10 in the respective end edges 5, 6, thus promoting ventilation of the absorbent article 1. The topsheet 10 in the respective end edges 5, 6 work as a gas permeable material, since gases can pass through it.

Furthermore, the ninth embodiment may be varied in accordance with the above described variations of the first embodiment. Thus, it may comprise more than one first ventilation component 14, whereby at least one first ventilation component 14 is positioned adjacent to the front end edge 5 and whereby the topsheet 10 has such an extension such that it at least partly extends over said at least one first ventilation component 14 in the Z direction of the article at the front end edge 5. Likewise, it may comprise more than one second ventilation component 15, whereby at least one second ventilation component 15 is positioned adjacent to the rear end edge 6 and whereby the topsheet 10 has such an extension such that it at least partly extends over said at least one second ventilation component 15 in the Z direction of the article at the rear end edge 5. In addition, the ninth embodiment may be varied such that it comprises one or more first ventilation components 14 in the front end section 2, but no second ventilation components 15 in the rear end section 3, or vice versa. Furthermore, the ninth embodiment may comprise one or more gas permeable panels 19 in the backsheet 8.

A tenth embodiment (not shown) of the absorbent article 1 according to the invention corresponds to the third embodiment shown in FIG. 3a except for concerning the fact that it does not comprise any third gas passage openings. Instead the tenth embodiment of the absorbent article 1 is gas permeable in such a way such that passage of gas out of the article 1 via the third ventilation components 21 is allowed when the article 1 is worn in that the topsheet 10 has such an extension that it at least partly extends over the third ventilation components 21 in the Z direction of the article at the side edges 7. Thus, the third ventilation components 21 are at least partly covered at the respective side edge 7 by the topsheet 10.

In the tenth embodiment, gas may pass into, within and out of the third ventilation components 21 and out of the article 1 through the topsheet 10 in the respective side edges 7, thus promoting ventilation of the absorbent article 1.

Furthermore, the tenth embodiment may be varied in accordance with the above described variations of the third embodiment. Thus, it may be varied such that at least one third ventilation component 21 is at least partly localized in each third margin section 13 and is located adjacent to the respective side edge 7. Then the topsheet 10 has such an extension such that it at least partly extends over said at least one third ventilation component 21 in the Z direction of the article at the side edges 7.

In a further variation (not shown) of the tenth embodiment, at least one third ventilation component 21 is localized partly in one third margin section 13 and partly in at least one of a first margin section 11 in the front end section 2 and a second margin section 12 in the rear end section 3 such that it extends from the third margin section 13 into at least one of the first and second margin sections 11, 12.

In still a further variation (not shown) of the tenth embodiment, at least one third ventilation component 21 is localized partly in one third margin section 13, partly in a first margin section 11 in the front end section 2 and partly in a second margin section 12 in the rear end section 3 such that it extends from the front end edge 5 to the rear end edge 6.

An eleventh embodiment (not shown) corresponds to any of the first, second, third or fourth embodiments or variations thereof except for concerning the fact that the eleventh embodiment does not comprise any gas passage openings. However, the eleventh embodiment comprises one or more gas permeable panels 19 in the backsheet 8 as described in the context of the first embodiment. Passage of gas into and out of the article 1 is allowed via the panel(s) 19, thus promoting ventilation of the article 1. Each gas permeable panel 19 may be positioned completely or partly underlying a ventilation component. Alternatively, it may be positioned in any other parts of the backsheet 8.

In another variant of any of the above described embodiments or variations thereof, the absorbent core 9 has such a shape and position in relation to the backsheet 8 that more than one first margin section 11 is formed in the front end section 2, and/or more than one second margin section 12 is formed in the rear end section 3 and/or more than one third margin section 13 is formed on each side of the absorbent core 9. Then there may be one or more first ventilation components 14 in at least one first margin section 11, and/or one or more second ventilation components 15 in at least one second margin section 12, and/or one or more third ventilation components 21 in at least one third margin section 13 on each side of the absorbent core 9.

Generally, the present invention relates to an absorbent article 1, such as a diaper, sanitary napkin, incontinence guard or panty liner, said article 1 having longitudinal (L) and transverse (T) directions and comprising:
a front end section 2, a rear end section 3 and a crotch section 4 interconnecting said end sections 2, 3;
a front end edge 5, a rear end edge 6 and two side edges 7;
a liquid impermeable backsheet 8;
an absorbent core 9 positioned overlaying a portion of said backsheet 8;
at least one margin section 11, 12, 13, whereby said at least one margin section 11, 12, 13 is located adjacent to at least one of said edges 5, 6, 7, whereby said backsheet 8 is not overlaid by said absorbent core 9 in said margin section(s) 11, 12, 13;
at least one ventilation component 14, 15, 21 constituted by a ventilation material, said at least one ventilation component 14, 15, 21 being positioned overlaying said backsheet 8 and arranged such that it is exclusively localized within one or more margin sections 11, 12, 13,
   wherein

the thickness of the ventilation material measured at 0.5 kPa is at least 1 mm, preferably at least 3 mm, more preferably at least 7 mm, most preferably 10 mm, the porosity of the ventilation material is >80%, preferably >90%, most preferably >95%,

the ratio between the thickness of the ventilation material measured at 5 kPa and the thickness of the ventilation material measured at 0.5 kPa is >0.5, preferably >0.75, most preferably >0.95, and

said at least one ventilation component 14, 15, 21 is arranged such that an associated spacing is created between the wearer's skin and the backsheet 8 when the absorbent article 1 is worn,

whereby passage of gas into, within and out of the said at least one ventilation component 14, 15, 21 is allowed, thus promoting ventilation of the absorbent article 1.

According to the invention, the ventilation material, i.e. the ventilation components 14, 15, 21, are only positioned in specific regions of the article 1 in which ventilation is most important to promote in order to reduce the risk of skin irritations and rashes. Thus, it is a cost effective way of promoting ventilation in specific regions of the article. In addition, since the ventilation material does not extend over the absorbent core, the risk of leakage from the absorbent core via the ventilation material is reduced.

### Test Methods

### Thickness Measurement

The thickness of a ventilation material at a specific pressure can be determined using Instron 4301 tensile tester (Instron, USA) with compression test software Instron series IX Automated Materials Tester-version 8.28.00, which measures the resisting force as the material is compressed between a moveable platen and a fixed base at a constant rate of 5 mm/min.

The pressure, at which the measurement is performed, is calculated using:

P=F/Aₚ

where:
P=pressure in Pascals
F=force on the platen in Newtons
Aₚ=area of the platen in square meters (for example 7.85e-5 m²)

The base used must be larger in size than the platen. Initial height is determined by running a compression test with no material between platen and base and recording the distance at contact. The initial height must be greater than the initial thickness of the sample so that the test starts out at zero pressure on the sample. The sample material can be of the same size as the platen or preferably larger, for example 5×5 cm.

The thickness is calculated using:

t=x₀−x

where:
t=thickness in millimetres of the sample for a specified pressure.
x=platen position from initial position in millimetres for a specified pressure.
x₀=initial platen position from the base in millimetres.

### Porosity Determination

Porosity of the ventilation material at 0.5 kPa is calculated using the equation:

$$ɛ = 1 - \frac{M}{{A}_{m} \cdot ( t \cdot 0.1 cm \text{/} mm )} \cdot \frac{1}{{ρ}_{fiber}}$$

where:
ε=porosity
M=mass of sample material in grams.
Aₘ=area of sample material in square centimetres.
ρ_{fiber}=fiber density in grams per cubic centimetre.
t=thickness in millimetres of the sample for the specified pressure.

For webs made with multiple fiber types, the web fiber density is the weight average of each individual fiber density:

ρ_{fiber Total}=wt %_{fiber1}·ρ_{fiber1}+wt %_{fiber2}·ρ_{fiber2}+ . . .

where:

$$wt \% = \frac{fiber weight in composition}{total compostition weight} \cdot 100 \%$$

When a foam material is being measured, ρ_{fiber} is the density of the material from which the foam is fabricated.

### EXAMPLES

The following are examples of materials that may be utilized as the ventilation material of the ventilation components according to the present invention.

### Ventilation Material A:

A spacer fabric made of 100% polyester (AirX, article no. 5683, 3 mm, Tytex)
M=1.4731 g
ρ_{fiber}=1.37 g/cm³
Aₘ=25 cm²
t_{0.5 kPa}=10.79 mm
t_{5 kPa}=10.30 mm

Calculations of porosity and thickness ratio using the preceding equations gives the following values for material A:

ε_{0.5 kPa}=0.96

t_{5 kPa}/t_{0.5 kPa}=0.95

### Ventilation Material B:

A spacer fabric made of 100% polyester (AirX, article no. 5911, 10 mm, Tytex)
M=0.5451 g
ρ_{fiber}=1.37 g/cm³
Aₘ=25 cm²
t_{0.5 kPa}=3.06 mm
t_{5 kPa}=2.88 mm

Calculations of porosity and thickness ratio using the preceding equations gives the following values for material B:

ε_{0.5 kPa}=0.93

t_{5 kPa}/t_{0.5 kPa}=0.94

### Ventilation Material C:

PP/PE bicomponent air-through nonwoven (ES-UB, 100 gsm, 20 dtex, ES FiberVisions)
M=0.2183 g
ρ_{fiber}=1.37 g/cm³
Aₘ=25 cm²
t_{0.5 kPa}=8.19 mm
t_{5 kPa}=2.44 mm

Calculations of porosity and thickness ratio using the preceding equations gives the following values for material C:

ε_{0.5 kPa}=0.99

t_{5 kPa}/t_{0.5 kPa}=0.30

The following are examples of materials that may be utilized as the gas permeable material of the gas permeable panels according to the present invention.

### Gas Permeable Material A:

30 gsm PP SMMS (WHITES107007, Fibertex A/S)

Air permeability according to the method WSP 70.1 (05): 59 m³/m²/min; STDEV=4, measurement performed at 200 Pa; 20 cm²

### Gas Permeable Material B:

17 gsm PP SSS, 1.1-1.3 dtex (Elite Soft, Fibertex A/S)

Air permeability according to the method WSP 70.1 (05): 248 m³/m²/min; STDEV=19, measurement performed at 200 Pa; 20 cm²

### Gas Permeable Material C:

30 gsm perforated biodegradable film (PVFX 271-P9, Poligof)

Air permeability according to the method WSP 70.1 (05): 100 m³/m²/min; STDEV=9, measurement performed at 200 Pa; 20 cm²

Gas Permeable Material D:

36 gsm perforated PE film (Aquidry Classic, Tredegar)

Air permeability according to the method WSP 70.1 (05): 181 m³/m²/min; STDEV=11, measurement performed at 200 Pa; 20 cm²

### Gas Permeable Material E:

15 gsm PP (4W H05-01; Fiberweb)

Air permeability according to the method WSP 70.1 (05): 364 m³/m²/min; STDEV=26, measurement performed at 200 Pa; 20 cm²

### Example of how to Measure Thickness of a Ventilation Material in a Product.

In a case where the ventilation material is inseparable from another material or materials in the product, for example a backsheet, and separating the materials is impossible without destroying the ventilation material, a good approximation of the thickness may be obtained by measuring the thickness of the composite containing the ventilation material and the inseparable materials together at the specified pressure, using the preceding method for thickness measurement, and further measuring the thickness of only the other material or materials at the same specified pressure. The thickness of the ventilation material may then be calculated by subtracting the measured thickness of the other material or materials from the value determined for the composite of the ventilation material and the other inseparable materials.

The invention has been described with reference to the embodied figures. However, the invention is not limited to the above-described embodiments alone. For example, although the invention has been described in detail with reference to a diaper, it is equally applicable to other types of absorbent articles, e.g. sanitary napkins, incontinence guards or panty liners. Features from one or more of the above embodiments or variants thereof may be combined as required, and the ultimate scope of the invention should be understood as being defined in the appended claims.

1. An absorbent article having longitudinal and transverse directions and comprising:
a front end section, a rear end section and a crotch section interconnecting said end sections;
a front end edge, a rear end edge and two side edges;
a liquid impermeable backsheet;
an absorbent core positioned overlaying a portion of said backsheet;
at least one margin section, whereby said at least one margin section is located adjacent to at least one of said edges, whereby said backsheet is not overlaid by said absorbent core in said at least one margin section; and
at least one ventilation component constituted by a ventilation material, said at least one ventilation component being positioned overlaying said backsheet, whereby the thickness of the ventilation material measured at 0.5 kPa is at least 1 mm, the porosity of the ventilation material is >80%, and the ratio between the thickness of the ventilation material measured at 5 kPa and the thickness of the ventilation material measured at 0.5 kPa is >0.5, wherein
said at least one ventilation component is arranged such that it is exclusively localized within one or more margin sections and does not overlap with, or extend over, the absorbent core, and
said at least one ventilation component is arranged such that an associated spacing is created between the wearer's skin and the backsheet when the absorbent article is worn,
whereby passage of gas into, within and out of said at least one ventilation component is allowed, thus promoting ventilation of the absorbent article.

2. An absorbent article according to claim 1, wherein
the article is gas permeable such that passage of gas out of the article via said at least one ventilation component is allowed when the article is worn.

3. An absorbent article according to claim 1, wherein
said article further comprises a liquid permeable topsheet, whereby at least one ventilation component and said absorbent core are positioned between said topsheet and said backsheet, and
at least one ventilation component positioned between said topsheet and said backsheet has at least one associated gas passage opening, whereby said at least one gas passage opening associated with a specific ventilation component is formed in any of said edges at a position adjacent to a margin section in which the respective ventilation component is localized,
whereby passage of gas out of the article via said at least one ventilation component positioned between said topsheet and said backsheet and said at least one associated gas passage opening is allowed, thus promoting ventilation of the absorbent article.

4. An absorbent article according to claim 3, wherein
said at least one associated gas passage opening is formed in any of said edges in that said topsheet and said backsheet are non-sealed in the area forming said at least one associated gas passage opening.

5. An absorbent article according to claim 3, wherein
said front end section comprises one first margin section adjacent to said front end edge;
at least one first ventilation component is localized in said first margin section and is positioned between said topsheet and said backsheet; and
said at least one first ventilation component has at least one associated first gas passage opening formed in said front end edge.

6. An absorbent article according to claim 5, wherein
at least one first ventilation component is positioned adjacent to at least one associated first gas passage opening.

7. An absorbent article according to claim 5, wherein
at least one first ventilation component is positioned spaced from its associated first gas passage opening(s).

8. An absorbent article according to claim 3, wherein
said rear end section comprises one second margin section adjacent to said rear end edge;
at least one second ventilation component is localized in said second margin section and positioned between said topsheet and said backsheet; and
said at least one second ventilation component has at least one associated second gas passage opening formed in said rear end edge.

9. An absorbent article according to claim 8, wherein
at least one second ventilation component is positioned adjacent to at least one associated second gas passage opening.

10. An absorbent article according to claim 8, wherein
that at least one second ventilation component is positioned spaced from its associated second gas passage opening(s).

11. An absorbent article according to claim 3, wherein
said crotch section comprises two third margin sections, whereby one third margin section is located adjacent to each side edge,
at least one third ventilation component is at least partly localized in each third margin section and is positioned between said topsheet and said backsheet; and
said at least one third ventilation component has at least one associated third gas passage opening formed in the respective side edge located adjacent to the respective third margin section in which the respective third ventilation component is at least partly localized.

12. An absorbent article according to claim 11, wherein
at least one third ventilation component is positioned adjacent to at least one associated third gas passage opening.

13. An absorbent article according to claim 11, wherein
at least one third ventilation component is positioned spaced from its associated third gas passage opening(s).

14. An absorbent article according to claim 11, wherein
at least one third ventilation component is localized partly in one third margin section and partly in at least one of a first margin section in said front end section and a second margin section in said rear end section such that it extends from the third margin section into at least one of said first and second margin sections.

15. An absorbent article according to claim 14, wherein
at least one third ventilation component is localized partly in one third margin section, partly in a first margin section in said front end section and partly in a second margin section in said rear end section, whereby it extends from a first gas passage opening formed in said front end edge to a second gas passage opening formed in said rear end edge.

16. An absorbent article according to claim 1, wherein
said article further comprises a liquid permeable topsheet, whereby at least one ventilation component and said absorbent core are positioned between said topsheet and said backsheet,
at least one ventilation component, which is positioned between said topsheet and said backsheet, has an associated gas permeable window, whereby said gas permeable window associated with a specific ventilation component is formed in any of said edges at a position adjacent to a margin section in which the respective ventilation component is localized, and whereby said gas permeable window is formed by a gas permeable material being arranged between the end of the topsheet and the end of the backsheet at said position, and
the gas permeable material of said gas permeable window has an air permeability of at least 50 m³/m²/min,
whereby passage of gas out of the article via said at least one ventilation component, which is positioned between said topsheet and said backsheet, and said associated gas permeable window is allowed, thus promoting ventilation of the absorbent article.

17. An absorbent article according to claim 16, wherein
said front end section comprises one first margin section adjacent to said front end edge;
at least one first ventilation component is localized in said first margin section and is positioned between said topsheet and said backsheet;
said at least one first ventilation component has one associated first gas permeable window formed in said front end edge; and
said at least one first ventilation component is positioned adjacent to its associated first gas permeable window.

18. An absorbent article according to claim 16, wherein
said rear end section comprises one second margin section adjacent to said rear end edge;
at least one second ventilation component is localized in said second margin section and is positioned between said topsheet and said backsheet; and
said at least one second ventilation component has one associated second gas permeable window formed in said rear end edge; and
said at least one second ventilation component is positioned adjacent to its associated second gas permeable window.

19. An absorbent article according to claim 18, wherein
said crotch section comprises two third margin sections, whereby one third margin section is located adjacent to each side edge,
at least one third ventilation component is at least partly localized in each third margin section and is positioned between said topsheet and said backsheet; and
said at least one third ventilation component has one associated third gas permeable window formed in the respective side edge located adjacent to the respective third margin section in which the respective third ventilation component is localized; and
said at least one third ventilation component is positioned adjacent to its associated third gas permeable window.

20. An absorbent article according to claim 19, wherein
at least one third ventilation component is localized partly in one third margin section and partly in at least one of a first margin section in said front end section and a second margin section in said rear end section such that it extends from the third margin section into at least one of said first and second margin sections.

21. An absorbent article according to claim 20, wherein
at least one third ventilation component is localized partly in one third margin section, partly in a first margin section in said front end section and partly in a second margin section in said rear section such that it extends from a first gas permeable window formed in said front end edge to a second gas permeable window formed in said rear end edge.

22. An absorbent article according to claim 1, wherein
said article further comprises a liquid permeable topsheet, whereby said absorbent core is positioned between said topsheet and said backsheet, and
at least one ventilation component is positioned overlaying said topsheet so as to be in contact with the wearer's skin during use of the article.

23. An absorbent article according to claim 22, wherein
said front end section comprises one first margin section adjacent to said front end edge, and
at least one first ventilation component is localized in said first margin section and is positioned overlaying said topsheet.

24. An absorbent article according to claim 22, wherein
said rear end section comprises one second margin section adjacent to said rear end edge, and
at least one second ventilation component is localized in said second margin section and is positioned overlaying said topsheet.

25. An absorbent article according to claim 22, wherein
said crotch section comprises two third margin sections, whereby one third margin section is located adjacent to each side edge, and
at least one third ventilation component is at least partly localized in each third margin section and is positioned overlaying said topsheet.

26. An absorbent article according to claim 25, wherein
at least one third ventilation component is localized partly in one third margin section and partly in at least one of a first margin section in said front end section and a second margin section in said rear end section such that it extends from the third margin section into at least one of said first and second margin sections.

27. An absorbent article according to claim 26, wherein
at least one third ventilation component is localized partly in one third margin section, partly in a first margin section in said front end section and partly in a second margin section in said rear end section, such that it extends from said front end edge to said rear end edge.

28. An absorbent article according to claim 1, wherein
said article further comprises a liquid permeable topsheet, whereby at least one ventilation component and said absorbent core are positioned between said topsheet and said backsheet,
at least one ventilation component, which is positioned between said topsheet and said backsheet, is positioned adjacent to at least one edge located adjacent to a margin section in which the respective ventilation component is localized, and
said topsheet has such an extension such that it at least partly extends over said at least one ventilation component, which is positioned adjacent to at least one edge, in the Z direction of the article at said at least one edge.

29. An absorbent article according to claim 28, wherein
said front end section comprises one first margin section adjacent to said front end edge,
at least one first ventilation component is localized adjacent to said front end edge in said first margin section and is positioned between said topsheet and said backsheet, and
said topsheet has such an extension such that it at least partly extends over said at least one first ventilation component in the Z direction of the article at said front end edge.

30. An absorbent article according to claim 28, wherein
said rear end section comprises one second margin section adjacent to said rear end edge,
at least one second ventilation component is localized adjacent to said rear end edge in said second margin section and is positioned between said topsheet and said backsheet, and
said topsheet has such an extension such that it at least partly extends over said at least one second ventilation component in the Z direction of the article at said rear end edge.

31. An absorbent article according to claim 28, wherein
said crotch section comprises two third margin sections, whereby one third margin section is located adjacent to each side edge,
at least one third ventilation component is at least partly localized in each third margin section, is located adjacent to the respective side edge and is positioned between said topsheet and said backsheet, and
said topsheet has such an extension such that it at least partly extends over said at least one third ventilation component in the Z direction of the article at the respective side edge.

32. An absorbent article according to claim 31, wherein
at least one third ventilation component is localized partly in one third margin section and partly in at least one of a first margin section in said front end section and a second margin section in said rear end section such that it extends from the third margin section into at least one of said first and second margin sections.

33. An absorbent article according to claim 32, wherein
at least one third ventilation component is localized partly in one third margin section, partly in a first margin section in said front end section and partly in a second margin section in said rear end section, such that it extends from said front end edge to said rear end edge.

34. An absorbent article according to claim 1, wherein
said backsheet comprises at least one gas permeable panel of a gas permeable material, whereby passage of gas into and out of the article through said at least one gas permeable panel is allowed, thus promoting ventilation of the absorbent article.

35. An absorbent article according to claim 34, wherein
at least one gas permeable panel is positioned at least partly underlying at least one ventilation component, whereby passage of gas into said at least one ventilation component being positioned at least partly overlying a gas permeable panel in said backsheet is promoted.

36. An absorbent article according to claim 34, wherein
the gas permeable material of said at least one gas permeable panel has an air permeability of at least 50 m³/m²/min.

37. An absorbent article according to claim 1, wherein the thickness of the ventilation material measured at 0.5 kPa is 3 mm.

38. An absorbent article according to claim 1, wherein the thickness of the ventilation material measured at 0.5 kPa is 7 mm.

39. An absorbent article according to claim 1, wherein the thickness of the ventilation material measured at 0.5 kPa is 10 mm.

40. An absorbent article according to claim 1, wherein the porosity of the ventilation material is >90%.

41. An absorbent article according to claim 1, wherein the porosity of the ventilation material is >95%.

42. An absorbent article according to claim 1, wherein the ratio between the thickness of the ventilation material measured at 5 kPa and the thickness of the ventilation material measured at 0.5 kPa is >0.75.

43. An absorbent article according to claim 1, wherein the ratio between the thickness of the ventilation material measured at 5 kPa and the thickness of the ventilation material measured at 0.5 kPa is >0.95.

44. An absorbent article according to claim 16, wherein the gas permeable material of said gas permeable window has an air permeability of 150 m³/m²/min.

45. An absorbent article according to claim 16, wherein the gas permeable material of said gas permeable window has an air permeability of 300 m³/m²/min.

46. An absorbent article according to claim 16, wherein the gas permeable material of said gas permeable window has an air permeability of 450 m³/m²/min.

47. An absorbent article according to claim 1, wherein the absorbent article is one of a diaper, a sanitary napkin, an incontinence guard and a panty liner.
